SEQUENCE LISTING (8 sequences)

SEQ ID NO: 1 - DNA, 17 bp, Artificial Sequence
  Description of Artificial Sequence Synthetic primer
agcagggatggacctgg

SEQ ID NO: 2 - DNA, 17 bp, Artificial Sequence
  Description of Artificial Sequence Synthetic primer
cggtagaggagcaccaa

SEQ ID NO: 3 - DNA, 18 bp, Artificial Sequence
  Description of Artificial Sequence Synthetic primer
cagcctggcagccagaga

SEQ ID NO: 4 - DNA, 20 bp, Artificial Sequence
  Description of Artificial Sequence Synthetic primer
tctgtggctccatcaatgca

SEQ ID NO: 5 - DNA, 20 bp, Artificial Sequence
  Description of Artificial Sequence Synthetic primer
cgtggctgttgagcgaataa

SEQ ID NO: 6 - DNA, 21 bp, Artificial Sequence
  Description of Artificial Sequence Synthetic primer
tctcaccttttttgggccaat

SEQ ID NO: 7 - DNA, 22 bp, Artificial Sequence
  Description of Artificial Sequence Synthetic primer
tgctgactgcaacacaaagtgt

SEQ ID NO: 8 - DNA, 27 bp, Artificial Sequence
  Description of Artificial Sequence Synthetic primer
agctgacatgtatgatagaccattgtc

(19) United States
(12) Patent Application Publication
(10) Pub. No.: US 2011/0055943 A1
(43) Pub. Date: Mar. 3, 2011

(54) NOVEL THERAPEUTIC TARGETS IN INFLAMMATORY BOWEL DISEASE

(75) Inventors: Frank J. Gonzalez, Bethesda, MD (US); Lorin Johnson, Palo Alto, CA (US); Xiaochao Ma, Bethesda, MD (US)

(73) Assignee: SALIX PHARMACEUTICALS, LTD., Morrisville, NC (US)

(21) Appl. No.: 12/682,665

(22) PCT Filed: Oct. 16, 2008
(86) PCT No.: PCT/US08/80115

§ 371 Date: Nov. 18, 2010

Related U.S. Application Data
(60) Provisional application No. 60/999,234, filed on Oct. 17, 2007.

(51) Int. Cl. A61K 31/437; G01N 33/53; C12Q 1/68; C07D 491/22; A01K 67/027; C12N 5/10; C07K 16/00; C07K 16/28; A61P 1/00

(52) U.S. Cl. 800/18; 435/7.21; 435/6; 546/40; 514/279; 435/354; 530/389.1; 530/391.1

(57) ABSTRACT

The present invention relates to novel sequences for use in detection, diagnosis and treatment of bowl disease (BD). The invention provides BD-associated polynucleotide sequences whose expression is associated with BD. Provided herein are diagnostic compositions and methods for the detection of BD. The present invention provides monoclonal and polyclonal antibodies specific for the BD polypeptides. The present invention also provides diagnostic tools and therapeutic compositions and methods for screening, prevention and treatment of BD.

### RELATED APPLICATIONS

This application is a continuation of International Application PCT/US2008/080115 filed on Oct. 16, 2008 which claims the benefit of U.S. Provisional Application 60/999,234 filed Oct. 17, 2007. The entire contents of the aforementioned applications are hereby incorporated herein by reference.

### BACKGROUND

Xifaxan® (Rifaximin, RIFax) was approved by the FDA in 2004 for the treatment of travelers' diarrhea (Laustsen and Wimmett, 2005). RIFax was shown to be a general antibiotic that acts to inhibit bacterial RNA synthesis. The mechanism contributing to the beneficial effects of RIFax in chronic gastrointestinal disorders are not fully understood.

Accordingly, there is a need in the art to provide polynucleotide and polypeptide sequences involved in bowel disease (BD) and, in particular, in irritable bowel syndrome (IBS). There is also a need in the art to provide antigens (BD associated polypeptides) associated with a variety of BDs as targets for diagnostic and/or therapeutic compounds and compositions, including antibodies. These antigens would also be useful for drug discovery (e.g., small molecules) and for further characterization of cellular regulation, growth, and differentiation. There is also a need to identify and characterize genes and related proteins that may be useful as a drug or pharmaceutical target, or may play a role in preventing, ameliorating, or correcting dysfunctions or diseases.

### SUMMARY

In one aspect, presented herein are methods of screening a compound or a salt thereof that modulates a pregnane X receptor (PXR) protein or fragment thereof, comprising contacting the PXR receptor with one or more candidate compounds.

In one embodiment, the candidate compound comprises a rifamycin analog.

In a related embodiment, the rifamycin analog comprises rifaximin.

In one embodiment, modulates includes modulating the signal transduction induced by binding of PXR protein or fragment thereof to a rifamycin analog.

In one embodiment, the PXR receptor is associated with a membrane, is in a transgenic mouse, is in an assay plate, is in a cell, and/or is in an artificial membrane.

In one embodiment, the PXR protein comprises the an amino acid sequence represented by sequence accession number O75469; Q8SQ01; Q9R1A7; O54915; NP_{—}148934; NP_{—}003880; or NP_{—}071285 or a fragment or variant thereof or a nucleic acid represented by sequence accession no NM_{—}033013; NM_{—}009803; NM_{—}022002; NM_{—}003889; CS618137; CS618135; CS618133 or a fragment or variant thereof.

In another embodiment, CYP3A11, GSTA1, MRP2 and OATP2 were up-regulated.

In one embodiment, the method may further comprise pretreatment with one or more of rifaximin, a rifamycin analog or a candidate compound.

In one embodiment, pretreatment with the candidate compound does not affect the pharmacokinetics of the CYP3A substrate midazolam.

In another embodiment, pretreatment with the candidate compound increases a Cₘₐₓ and decreases a Tₘₐₓ of 1′-hydroxymidazolam.

In one embodiment, CYP3A11 increases from about 1 to about 4-fold compared to a control after treatment with the candidate compound.

In another embodiment, GSTA1 mRNA is up-regulated after candidate compound treatment.

In another embodiment, the up-regulation ranges from between about 65% to about 200%.

In one embodiment, there is an up-regulation of intestinal MRP2 mRNA following candidate compound treatment.

In one aspect, presented herein are kits for screening (I) a compound or a salt thereof that modulate a PXR receptor or fragment thereof, comprising a PXR receptor protein or an active fragment thereof and a rifamycin analog.

In one aspect, presented herein are medicaments for treatment of a PXR related disorder comprising a compound or a salt thereof that modulates a pregnane X receptor (PXR) protein or fragment thereof, or a compound or its salt that modulates signal transduction induced by binding of PXR protein or fragment thereof to a rifamycin analog.

In one aspect, presented herein are methods of treating, preventing, or alleviating a PXR related disorder in a subject comprising administering a compound or a salt thereof that modulates a pregnane X receptor (PXR) protein or fragment thereof.

In one embodiment, the compound or a salt thereof that modulates a pregnane X receptor (PXR) protein or fragment thereof is not rifaximin.

In one embodiment, modulates includes modulating the signal transduction induced by binding of PXR protein or fragment thereof to a rifamycin analog.

In another embodiment, the PXR receptor is associated with a membrane, is in a transgenic mouse, is in an assay plate, is in a cell, and/or is in an artificial membrane.

In one embodiment, the PXR protein comprises the an amino acid sequence represented by sequence accession number O75469; Q8SQ01; Q9R1A7; O54915; NP_{—}148934; NP_{—}003880; or NP_{—}071285 or a fragment or variant thereof or a nucleic acid represented by sequence accession no NM_{—}033013; NM_{—}009803; NM_{—}022002; NM_{—}003889; CS618137; CS618135; CS618133 or a fragment or variant thereof.

In one aspect, presented herein are compositions comprising a PXR protein agonist in an amount effective to produce a therapeutic effect.

In one aspect, presented herein are transgenic mice comprising a homozygous disruption of the endogenous pregnane X receptor (PXR) gene.

In another embodiment, the mouse comprises a human PXR gene.

In one aspect, presented herein are cells or tissues isolated from the transgenic mice described herein.

In one aspect, presented herein are methods of identifying an agent capable of modulating activity of a PXR gene or of a PXR gene expression product, comprising administering a putative agent to a transgenic mice described herein; administering the agent to a wild-type control mouse; and comparing a physiological response of the transgenic mouse with that of the control mouse; wherein a difference in the physiological response between the transgenic mouse and the control mouse is an indication that the agent is capable of modulating activity of the gene or gene expression product.

In one aspect, presented herein are methods of screening for a drug candidate having anti-inflammatory bowel disease activity comprising providing a cell that expresses a PXR gene encoded by a nucleic acid sequence selected from the group consisting of the sequences NM_{—}033013; NM_{—}009803; NM_{—}022002; NM_{—}003889; CS618137; CS618135; CS618133 or. O75469; Q8SQ01; Q9R1A7; O54915; NP_{—}148934; NP_{—}003880; NP_{—}071285, or a fragments or variants thereof; contacting the cell with a drug candidate; and monitoring an effect of the drug candidate on an expression of the BD polynucleotide in the tissue sample.

In one aspect, presented herein are isolated antibodies or antigen binding fragment thereof that binds to a PXR polypeptide.

In one embodiment, the antibody or fragment thereof is attached to a solid support; wherein the antibody is a monoclonal antibody; wherein the antibody is a polyclonal antibody; and/or wherein the antibody or fragment thereof further comprises a detectable label.

The present invention is based, in part, on the finding that susceptibility to BD is strongly associated with genetic variation in the PXR gene, a member of the nuclear receptor family. The PXR is a nuclear receptor that regulates genes involved in xenobiotic and limited antibiotic deposition and detoxication. The invention is also based, in part, on the RIFax specific activation of human PXR. PXR is an integral component of the body's defense mechanism involved in endogenous and xenobiotic detoxication (Kliewer et al., 2002). PXR is activated by a broad spectrum of xenobiotics including prescription drugs, herbal supplements, pesticides, endocrine disruptors and other environmental contaminants (Carnahan and Redinbo, 2005). PXR activation regulates a number of genes involved in the metabolism and excretion of xenobiotics including toxic chemicals (Kliewer, 2003; Rosenfeld et al., 2003; Sonoda et al., 2005). Disclosed herein is also a novel animal model, PXR-humanized mice (hPXR), in which an entire human PXR gene was re-introduced into a Pxr-null background (Ma et al., 2007).

The present invention provides methods for screening for compositions that modulate inflammatory bowel disease. The present invention also provides methods for screening for compositions which modulate inflammatory bowel disease. Also provided herein are methods of inhibiting inflammation of the bowel and related tissues and organs. Methods of treatment of inflammatory bowel disease are also provided herein.

The present disclosure generally relates to transgenic animals, as well as to compositions and methods relating to the characterization of gene function.

In one aspect, a method of screening drug candidates comprises providing a cell line that expresses an inflammatory bowel disease-associated (BD) gene or fragments thereof. Certain embodiments of BD genes are genes that are differentially expressed in BD. Certain embodiments of BD genes used in the methods herein include, but are not limited to the nucleic acids selected from NCBI Accession Nos.: NM_{—}033013; NM_{—}009803; NM_{—}022002; NM_{—}003889; CS618137; CS618135; CS618133 or fragments thereof corresponding to the human mRNAs generated therefrom). The methods further include adding a drug candidate to the cell and determining the effect of the drug candidate on the expression, binding, behavior of the BD genes.

In one embodiment, the method of screening drug candidates includes comparing the level of expression in the absence of the drug candidate to the level of expression in the presence of the drug candidate.

Also provided herein is a method of screening for a bioactive agent capable of binding to a BD protein (BDP) comprising combining the BDP and a candidate bioactive agent, and determining the binding of the candidate agent to the BDP.

Further provided herein is a method for screening for a bioactive agent capable of modulating the activity of a BDP. In one embodiment, the method comprises combining the BDP and a candidate bioactive agent, and determining the effect of the candidate agent on the bioactivity of the BDP.

Also provided is a method of evaluating the effect of a candidate BD drug comprising administering the drug to a subject and removing a cell sample from the subject. The expression profile of the cell is then determined. This method may further comprise comparing the expression profile of the subject to an expression profile of a healthy individual.

A BD protein, for example, comprises a protein selected from the group consisting of the sequences outlined in NCBI Accession Nos. O75469; Q8SQ01; Q9R1A7; O54915; NP_{—}148934; NP_{—}003880; NP_{—}071285. BDP proteins, as used herein, include, for example, proteins in the signal transduction pathway after activation by a ligand.

BD protein, for example, comprises a protein encoded by a nucleic acid selected from the group of sequences outlined in NCBI Accession Nos.: NM_{—}033013; NM_{—}009803; NM_{—}022002; NM_{—}003889; CS618137; CS618135; CS618133; corresponding to the human mRNAs generated therefrom.

Moreover, provided herein is a biochip comprising a nucleic acid segment which encodes a BD protein, nucleic acid selected from the group of sequences outlined in NCBI accession nos.: NM_{—}033013; NM_{—}009803; NM_{—}022002; NM_{—}003889; CS618137; CS618135; CS618133; corresponding to the human mRNAs generated therefrom.

Also provided herein is a method for diagnosing or determining the propensity to BDs, by sequencing at least one BD gene of a subject. In yet another aspect of the invention, a method is provided for determining BD including gene copy numbers in a subject.

In some embodiments, the polynucleotide, or its complement or a fragment thereof, further comprises a detectable label, is attached to a solid support, is prepared at least in part by chemical synthesis, is an antisense fragment, is single stranded, is double stranded or comprises a microarray.

Provided herein are isolated polypeptides, encoded within an open reading frame of a BD sequence selected from the group consisting of the polynucleotide sequences described infra or the complement. Provided herein are isolated polypeptides, wherein the polypeptides comprise the amino acid sequence encoded by a polynucleotide selected from the group consisting of sequences described infra. Provided herein are isolated polypeptides, wherein the polypeptides comprise the amino acid sequence encoded by a polypeptide selected from the group consisting of sequences described infra. In certain embodiments, the polypeptide or fragment thereof may be attached to a solid support. In one embodiment, provided herein are isolated antibodies (monoclonal or polyclonal) or antigen binding fragments thereof, that bind to such a polypeptide. The isolated antibody or antigen binding fragment thereof may be attached to a solid support, or further comprises a detectable label.

In one embodiment, provided herein are methods of screening for anti-BD activity comprising: (a) providing a cell that expresses a BD associated gene encoded by a nucleic acid sequence selected from the group consisting of the BD sequences of Accession Nos.: NM_{—}033013; NM_{—}009803; NM_{—}022002; NM_{—}003889; CS618137; CS618135; CS618133 or Accession Nos. O75469; Q8SQ01; Q9R1A7; O54915; NP_{—}148934; NP_{—}003880; NP_{—}071285 or fragment thereof; (b) contacting a tissue sample derived from a BD cell with an anti-BD drug candidate (e.g., candidate composition); (c) monitoring an effect of the anti-BD drug candidate on an expression of the BD polynucleotide in the tissue sample, and optionally (d) comparing the level of expression in the absence of the drug candidate to the level of expression in the presence of the drug candidate. The drug candidate may be an inhibitor of transcription, a G-protein coupled receptor antagonist, a growth factor antagonist, a serine-threonine kinase antagonist, a tyrosine kinase antagonist.

In one embodiment, compounds include, for example rifamycin derivatives bearing a heterocyclic ring which is condensed at the 3,4-positions. Exemplary compositions include, for example, those compounds disclosed in South African Pat. No. 68/0903 (pyrrolo[5,4-c]rifamycin SV derivatives), German Patent Publications Nos. 2,739,671 and 2,739,623 (imidazo[5,4-c]rifamycin SV compounds which bear substituent at the positions 1 and 2). Thiazolo[5,4-c]rifamycin SV (rifamycin P) derivatives are reported in the German Patent Publication No. 2,741,066. Other rifamycin analogues include, for example, —O and —P, which are well know to one of skill in the art and may be found along with other compositions and derivatives thereof, for example, in U.S. Pat. No. 4,341,785 or U.S. Pat. No. 4,200,574.

Provided herein are methods for screening for a bioactive agent capable of modulating the activity of a BD protein (BDP), wherein the BDP is encoded by a nucleic acid comprising a nucleic acid sequence described infra, comprising: combining the BDP and a candidate bioactive agent; and determining the effect of the candidate agent on the bioactivity of the BDP. According to the method the bioactive agent may affect the expression of the BD protein (BDP); affect the activity of the BD protein (BDP).

The invention provides monoclonal antibodies that preferentially bind to a BD protein (BDP), wherein the BD protein selected from those described herein; optionally linked to a therapeutic agent; or humanized. Kits and pharmaceutical compositions for detecting a presence or an absence of BD cells in a subject, and comprising such antibodies are also provided.

The present disclosure provides transgenic cells comprising a disruption in a PXR gene. The transgenic cells of the present disclosure are comprised of any cells capable of undergoing homologous recombination. Preferably, the cells of the present disclosure are stem cells and more preferably, embryonic stem (ES) cells, and most preferably, murine ES cells. According to one embodiment, the transgenic cells are produced by introducing a targeting construct into a stem cell to produce a homologous recombinant, resulting in a mutation of the PXR gene. In another embodiment, the transgenic cells are derived from the transgenic animals described below. The cells derived from the transgenic animals include cells that are isolated or present in a tissue or organ, and any cell lines or any progeny thereof.

The present disclosure also provides a targeting construct and methods of producing the targeting construct that when introduced into stem cells produces a homologous recombinant. In one embodiment, the targeting construct of the present disclosure comprises first and second polynucleotide sequences that are homologous to the PXR gene. The targeting construct also comprises a polynucleotide sequence that encodes a selectable marker that is preferably positioned between the two different homologous polynucleotide sequences in the construct. The targeting construct may also comprise other regulatory elements that may enhance homologous recombination.

The present disclosure further provides non-human transgenic animals and methods of producing such non-human transgenic animals comprising a disruption in a PXR gene. The transgenic animals of the present disclosure include transgenic animals that are heterozygous and homozygous for a mutation in the PXR gene. In one aspect, the transgenic animals of the present disclosure are defective in the function of the PXR gene. In another aspect, the transgenic animals of the present disclosure comprise a phenotype associated with having a mutation in a PXR gene.

The present disclosure also provides methods of identifying agents capable of affecting a phenotype of a transgenic animal. For example, a putative agent is administered to the transgenic animal and a response of the transgenic animal to the putative agent is measured and compared to the response of a “normal” or wild type mouse, or alternatively compared to a transgenic animal control (without agent administration). The disclosure further provides agents identified according to such methods. The present disclosure also provides methods of identifying agents useful as therapeutic agents for treating conditions associated with a disruption of the PXR gene.

The present disclosure further provides a method of identifying agents having an effect on PXR expression or function. The method includes administering an effective amount of the agent to a transgenic animal, for example, a mouse. The method includes measuring a response of the transgenic animal, for example, to the agent, and comparing the response of the transgenic animal to a control animal, which may be, for example, a wild-type animal or alternatively, a transgenic animal control. Compounds that may have an effect on PXR expression or function may also be screened against cells in cell-based assays, for example, to identify such compounds. Cells may be cells derived from the PXR transgenic mouse described herein.

The disclosure also provides cell lines comprising nucleic acid sequences of a PXR gene. Such cell lines may be capable of expressing such sequences by virtue of operable linkage to a promoter functional in the cell line. In one embodiment, the expression of the PXR gene sequence is under the control of an inducible promoter. Also provided are methods of identifying agents that interact with the PXR gene, comprising the steps of contacting the PXR gene with an agent and detecting an agent/PXR gene complex. Such complexes can be detected by, for example, measuring expression of an operably linked detectable marker.

The disclosure further provides methods of treating diseases or conditions associated with a disruption in a PXR gene, and more particularly, to a disruption in the expression or function of the PXR gene. In one embodiment, methods of the present disclosure involve treating diseases or conditions associated with a disruption in the PXR genes expression or function, including administering to a subject in need, a therapeutic agent that effects PXR expression or function. In accordance with this embodiment, the method comprises administration of a therapeutically effective amount of a natural, synthetic, semi-synthetic, or recombinant PXR gene, PXR gene products or fragments thereof as well as natural, synthetic, semi-synthetic or recombinant analogs.

The present disclosure further provides methods of treating diseases or conditions associated with disrupted targeted gene expression or function, wherein the methods comprise detecting and replacing through gene therapy mutated PXR genes.

In another embodiment, the phenotype (or phenotypic change) associated with a disruption in the PXR gene is used to predict the likely effects and side effects of a drug that antagonizes the PXR gene product. In this embodiment, the mouse is used to evaluate the gene as a “druggable target” e.g., to determine whether the development of drugs that target the PXR gene product would be a worthwhile focus for pharmaceutical research.

Other embodiments are disclosed infra.

### BRIEF DESCRIPTION OF THE FIGURES

FIG. 1 depicts LC-MS/MS analysis of RIF and RIFax. (A) Structure of RIF; (B) Structure of RIFax; (C) Typical chromatogram of RIF and RIFax. RIF and RIFax were detected by LC-MS/MS, m/z 823.5/791.5 for RIF (1), and m/z 786.3/754.5 for RIFax (2).

FIG. 2 depicts metabolic profiles and intestinal tract distribution of RIF and RIFax in mice, following a single dose of 10 mg/kg RIF or RIFax treatment. (A) Concentration-time plots of serum RIF and RIFax after oral treatment. Data are expressed as means±SD, n=3 at each time point. (B, C, D) Time course of RIF and RIFax in small intestine (S. intestine), cecum, and colon after oral treatment. The content in small intestine (B), cecum (C), and colon (D) were collected separately at 1.5, 3, 6, 9, 12, 24, and 48 h after the administration. RIF and RIFax were extracted from the content of intestinal tract, and analyzed by LC-MS/MS. Data are expressed as means±SD, n=3 at each time point. (E) RIFax Cₘₐₓ comparison among WT, Pxr-null and hPXR mice after oral treatment. Data are expressed as means (n=3). (F) Concentration-time plots of serum RIFax by i.v., i.p., and p.o. treatment. Data are expressed as means (n=3). (G) Concentration-time plots of serum RIFax and RIF after i.p. injection. Data are expressed as means (n=3). (H) Concentration-time plots of serum RIFax and RIF after i.v. injection. Data are expressed as means (n=3).

FIG. 3 depicts the effect of RIF and RIFax on PXR target genes in small intestine (S. intestine) and liver of WT, Pxr-null and hPXR mice. Mice were treated orally with 25 mg/kg of RIF or RIFax for 3 days and expression of CYP3A11, GSTA1, MRP2, and OATP2 were analyzed by qPCR. Values were quantified using the Comparative CT method, and samples normalized to β-actin. Data are expressed as means±SD, n=3. *p<0.05 compared with control. (A) Effect of RIFax on PXR target genes in S. intestine of hPXR mice; (B) Effect of RIFax on PXR target genes in S. intestine of WT mice; (C) Effect of RIFax on PXR target genes in S. intestine of Pxr-null mice; (D) Effect of RIF on PXR target genes in S. intestine of hPXR mice; (E) Effect of RIF on PXR target genes in liver of hPXR mice; (F) Effect of RIFax on PXR target genes in liver of hPXR mice.

FIG. 4 depicts cell-based reporter assay to determine RIFax activation of various xenobiotic nuclear receptors. Data are expressed as means±SD, n=3. *p<0.05 compared with control. (A) Cell-based reporter assay of RIFax on human PXR activation. 10 μM RIF; 1, 10, 100 μM RIFax were added separately to the culture medium. DMSO was used as vehicle. Activation of PXR was determined by measuring the firefly luciferase activity 24 h later, followed by normalization of the luciferase activity by protein concentrations. (B) Cell-based reporter assay of RIFax on human PXR, CAR, PPARα, PPARγ, and FXR activation. 10 μM RIFax was added to the culture medium for 24 h incubation. RIF (10 μM), TCPOBOP (250 nM), Wy-14,643 (10 μM), rosiglitazone (10 μM), and GW4064 (25 μM) were used as positive controls respectively for human PXR, CAR, PPARα, PPARγ, and FXR. DMSO was used as vehicle. A standard dual luciferase assay was used and normalized to a cotransfected control reporter.

### DETAILED DESCRIPTION

Rifaximin (RIFax), a rifamycin analogue approved for the treatment of travelers' diarrhea, is also beneficial in the treatment of gastrointestinal disorders or BDs. However, the mechanisms contributing to the effects of rifaximin on chronic gastrointestinal disorders are not fully understood. The present invention is based, in part, on rifaximin's role in specific activation of the human pregnane X receptor (PXR), a nuclear receptor that regulates a genes involved in xenobiotic and limited endobiotic deposition and detoxication. The present invention is also based, in part on the creation of PXR-humanized (hPXR), Pxr-null, and wild-type mice.

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

The term “effective amount” includes an amount effective, at dosages and for periods of time necessary, to achieve the desired result, e.g., sufficient to treat or prevent BD in a patient or subject. An effective amount of a PXR receptor modulator may vary according to factors such as the disease state, age, and weight of the subject, and the ability of a PXR receptor modulator to elicit a desired response in the subject. Dosage regimens may be adjusted to provide the optimum therapeutic response. An effective amount is also one in which any toxic or detrimental effects (e.g., side effects) of a PXR receptor modulator are outweighed by the therapeutically beneficial effects.

“Ameliorate,” “amelioration,” “improvement” or the like refers to, for example, a detectable improvement or a detectable change consistent with improvement that occurs in a subject or in at least a minority of subjects, e.g., in at least about 2%, 5%, 10%, 15%, 20%, 25%, 30%, 40%, 50%, 60%, 70%, 75%, 80%, 85%, 90%, 95%, 98%, 100% or in a range between about any two of these values. Such improvement or change may be observed in treated subjects as compared to subjects not treated with rifaximin, where the untreated subjects have, or are subject to developing, the same or similar disease, condition, symptom or the like. Amelioration of a disease, condition, symptom or assay parameter may be determined subjectively or objectively, e.g., self assessment by a subject(s), by a clinician's assessment or by conducting an appropriate assay or measurement, including, e.g., a quality of life assessment, a slowed progression of a disease(s) or condition(s), a reduced severity of a disease(s) or condition(s), or a suitable assay(s) for the level or activity(ies) of a biomolecule(s), cell(s) or by detection of BD episodes in a subject. Amelioration may be transient, prolonged or permanent or it may be variable at relevant times during or after a PXR receptor modulator is administered to a subject or is used in an assay or other method described herein or a cited reference, e.g., within timeframes described infra, or about 1 hour after the administration or use of a PXR receptor modulator to about 28 days, or 1, 3, 6, 9 months or more after a subject(s) has received such treatment.

The “modulation” of, e.g., a symptom, level or biological activity of a molecule, or the like, refers, for example, that the symptom or activity, or the like is detectably increased or decreased. Such increase or decrease may be observed in treated subjects as compared to subjects not treated with a PXR receptor modulator, where the untreated subjects have, or are subject to developing, the same or similar disease, condition, symptom or the like. Such increases or decreases may be at least about 2%, 5%, 10%, 15%, 20%, 25%, 30%, 40%, 50%, 60%, 70%, 75%, 80%, 85%, 90%, 95%, 98%, 100%, 150%, 200%, 250%, 300%, 400%, 500%, 1000% or more or within any range between any two of these values. Modulation may be determined subjectively or objectively, e.g., by the subject's self assessment, by a clinician's assessment or by conducting an appropriate assay or measurement, including, e.g., quality of life assessments or suitable assays for the level or activity of molecules, cells or cell migration within a subject. Modulation may be transient, prolonged or permanent or it may be variable at relevant times during or after a PXR receptor modulator is administered to a subject or is used in an assay or other method described herein or a cited reference, e.g., within times descried infra, or about 1 hour of the administration or use of a PXR receptor modulator to about 3, 6, 9 months or more after a subject(s) has received a PXR receptor modulator.

The term “modulate” may also refer to increases or decreases in the activity of a cell in response to exposure to a PXR receptor modulator, e.g., the inhibition of proliferation and/or induction of differentiation of at least a sub-population of cells in an animal such that a desired end result is achieved, e.g., a therapeutic result of PXR receptor modulator used for treatment may increase or decrease over the course of a particular treatment.

The term “obtaining” as in “obtaining a PXR receptor modulator” is intended to include purchasing, synthesizing or otherwise acquiring a PXR receptor modulator.

The phrases “parenteral administration” and “administered parenterally” as used herein includes, for example, modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The language “a prophylactically effective amount” of a compound refers to an amount of a PXR receptor modulator which is effective, upon single or multiple dose administration to the subject, in preventing or treating BD.

The term “pharmaceutical agent composition” (or agent or drug) as used herein refers to a chemical compound, composition, agent or drug capable of inducing a desired therapeutic effect when properly administered to a patient. It does not necessarily require more than one type of ingredient.

The phrases “systemic administration,” “administered systemically,” “peripheral administration,” and “administered peripherally,” as used herein mean the administration of a PXR receptor modulator, drug or other material, such that it enters the subject's system and, thus, is subject to metabolism and other like processes, for example, subcutaneous administration.

The language “therapeutically effective amount” of a PXR receptor modulator refers to an amount of a PXR receptor modulator which is effective, upon single or multiple dose administration to the subject, in inhibiting the bacterial growth and/or invasion, or in decreasing symptoms, such as BD episodes, relating to bacterial growth in a subject. “Therapeutically effective amount” also refers to the amount of a therapy (e.g., a composition comprising a PXR receptor modulator), which is sufficient to reduce the severity of BD in a subject.

As used herein, the terms “prevent,” “preventing,” and “prevention” refer to the prevention of the recurrence, onset, or development BD episodes or more symptoms of BD. Preventing includes protecting against the occurrence and severity of BD episodes.

As used herein, the term “prophylactically effective amount” refers to the amount of a therapy (e.g., a composition comprising a PXR receptor modulator) which is sufficient to result in the prevention of the development, recurrence, or onset of BD episodes or to enhance or improve the prophylactic effect(s) of another therapy.

As used herein, “subject” includes organisms which are capable of suffering from a bowel disorder or other disorder treatable by a PXR modulator or who could otherwise benefit from the administration of a PXR modulator as described herein, such as human and non-human animals. Preferred human animals include human subjects. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc.

The present invention is directed to a number of sequences associated with BD.

Described herein are methods of treating subjects suffering from or susceptible to gastrointestinal disorders or bowel disease by administering a PXR modulator formulation to a subject. The administration a PXR modulator formulation, as described herein increases the efficacy of treatment in subjects having gastrointestinal disorders. Exemplary gastrointestinal disorders and bowel diseases (PXR related disorder or PXR protein related disorder) that may be treated using the methods of the invention include, but are not limited to, for example, irritable bowel syndrome, Crohn's disease, traveler's diarrhea, ulcerative colitis, enteritis, small intestinal bacterial overgrowth, chronic pancreatitis, pancreatic insufficiency, colitis or hepatic encephalopathy. Subjects who may particularly benefit from this treatment include those who are or may be susceptible to BDs. For example, subjects who have recently had a food borne illness. In one embodiment, the bowel disease comprises hepatic encephalopathy. In one embodiment, a BD comprises one or more of inflammatory bowel disease (IBD), Crohn's disease, hepatic encephalopathy, enteritis, colitis, irritable bowel syndrome (IBS), fibromyalgia (FM), chronic fatigue syndrome (CFS), depression, attention deficit/hyperactivity disorder (ADHD), multiple sclerosis (MS), systemic lupus erythematosus (SLE), travelers' diarrhea, small intestinal bacterial overgrowth, chronic pancreatitis, or pancreatic insufficiency.

Accordingly, the present invention provides nucleic acid and protein sequences that are associated with BD, herein termed “BD associated” or “BD” sequences. In addition, the BD genes may be involved in other diseases such as, but not limited to, diseases associated with aging or neurodegeneration. “Association” in this context means that the nucleotide or protein sequences are either differentially expressed, activated, inactivated or altered in BDs as compared to normal tissue. As outlined below, BD sequences include those that are up-regulated (e.g., expressed at a higher level), as well as those that are down-regulated (e.g., expressed at a lower level), in BDs. BD sequences also include sequences that have been altered (e.g., truncated sequences or sequences with substitutions, deletions or insertions, including point mutations) and show either the same expression profile or an altered profile. In one embodiment, the BD sequences are from humans; however, as will be appreciated by those in the art, BD sequences from other organisms may be useful in animal models of disease and drug evaluation; thus, other BD sequences are provided, from vertebrates, including mammals, including rodents (rats, mice, hamsters, guinea pigs, etc.), primates, and farm animals (including sheep, goats, pigs, cows, horses, etc). In some cases, prokaryotic BD sequences may be useful. BD sequences from other organisms may be obtained using the techniques outlined below.

BD sequences include both nucleic acid and amino acid sequences. In one embodiment, the BD sequences are recombinant nucleic acids. In one embodiment, the BD sequences are nucleic acids. As will be appreciated by those in the art and is more fully outlined below, BD sequences are useful in a variety of applications, including diagnostic applications, which will detect naturally occurring nucleic acids, as well as screening applications; for example, biochips comprising nucleic acid probes to the BD sequences can be generated. In the broadest sense, use of “nucleic acid,” “polynucleotide” or “oligonucleotide” or equivalents herein means at least two nucleotides covalently linked together. In some embodiments, an oligonucleotide is an oligomer of 6, 8, 10, 12, 20, 30 or up to 100 nucleotides. A “polynucleotide” or “oligonucleotide” may comprise DNA, RNA, PNA or a polymer of nucleotides linked by phosphodiester and/or any alternate bonds.

The term “label” refers, for example, to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical, chemical, or any other appropriate means. The term “label” is used to refer to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The label may also be a compound that is a member of a binding pair, the other member of which bears a detectable physical property.

As used herein, a “biological sample”, refers, for example, to a sample of tissue or fluid isolated from a subject, including but not limited to, for example, blood, plasma, serum, spinal fluid, lymph fluid, skin, respiratory, intestinal and genitourinary tracts, tears, saliva, milk, cells (including but not limited to blood cells), BDs, organs, and also samples of in vitro cell culture constituents.

The term “biological sources” as used herein refers, for example, to the sources from which the target polynucleotides are derived. The source can be of any form of “sample” as described above, including but not limited to, cell, tissue or fluid. “Different biological sources” can refer to different cells/tissues/organs of the same individual, or cells/tissues/organs from different individuals of the same species, or cells/tissues/organs from different species.

The term “gene” refers, for example, to (a) a gene containing at least one of the DNA sequences disclosed herein; (b) any DNA sequence that encodes the amino acid sequence encoded by the DNA sequences disclosed herein and/or; (c) any DNA sequence that hybridizes to the complement of the coding sequences disclosed herein. Preferably, the term includes coding as well as noncoding regions, and preferably includes all sequences necessary for normal gene expression including promoters, enhancers and other regulatory sequences.

A “PXR gene” refers, for example, to a comprising the sequence identified in Genebank as Accession No. Accession Nos.: NM_{—}033013; NM_{—}009803; NM_{—}022002; NM_{—}003889; CS618137; CS618135; CS618133 or Accession Nos. O75469; Q8SQ01; Q9R1A7; O54915; NP_{—}148934; NP_{—}003880; NP_{—}071285. “Disruption” of a PXR gene occurs when a fragment of genomic DNA locates and recombines with an endogenous homologous sequence. These sequence disruptions or modifications may include insertions, missense, frameshift, deletion, or substitutions, or replacements of DNA sequence, or any combination thereof. Insertions include the insertion of entire genes, which may be of animal, plant, fungal, insect, prokaryotic, or viral origin. Disruption, for example, can alter or replace a promoter, enhancer, or splice site of a PXR gene, and can alter the normal gene product by inhibiting its production partially or completely or by enhancing the normal gene product's activity.

The term, “transgenic cell”, refers, for example, to a cell containing within its genome a PXR gene that has been disrupted, modified, altered, or replaced completely or partially by the method of gene targeting.

The term “transgenic animal” refers, for example, to an animal that contains within its genome a specific gene that has been disrupted by the method of gene targeting. The transgenic animal includes both the heterozygote animal (e.g., one defective allele and one wild-type allele) and the homozygous animal (e.g., two defective alleles).

As used herein, the terms “selectable marker” or “positive selection marker” refers, for example, to a gene encoding a product that enables only the cells that carry the gene to survive and/or grow under certain conditions. For example, plant and animal cells that express the introduced neomycin resistance (Ned) gene are resistant to the compound G418. Cells that do not carry the Ned gene marker are killed by G418. Other positive selection markers will be known to those of skill in the art.

A “host cell” includes a subject cell or cell culture that can be or has been a recipient for vector(s) or for incorporation of nucleic acid molecules and/or proteins. Host cells include progeny of a single host cell, and the progeny may not necessarily be completely identical (in morphology or in total DNA complement) to the original parent due to natural, accidental, or deliberate mutation. A host cell includes cells transfected with the constructs of the present disclosure.

The term “modulates” as used herein refers, for example, to the inhibition, reduction, increase or enhancement of a PXR function, expression, activity, or alternatively a phenotype associated with a disruption in a PXR gene. In reference to a PXR receptor, module may also include alters the binding property of, binds to, activates, associates with, and/or inhibits the activity of the receptor or the interaction of a molecule and the receptor.

The term “ameliorates” refers, for example, to a decreasing, reducing or eliminating of a condition, disease, disorder, or phenotype, including an abnormality or symptom associated with a disruption in a PXR gene.

The term “abnormality” refers, for example, to any disease, disorder, condition, or phenotype in which a disruption of a PXR gene is implicated, including pathological conditions.

A rifamycin class antibiotic is, for example, a compound having the structure of Formula I:

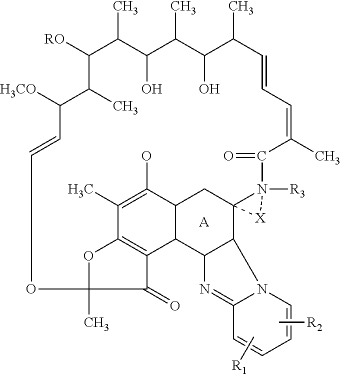

wherein A may be the structure A₁:

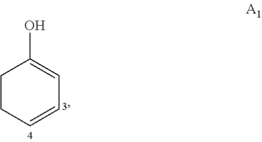

or the structure A₂

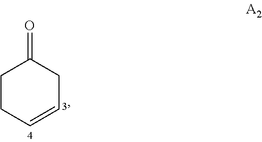

wherein, -x- is a covalent chemical bond or nil; R is hydrogen or acetyl;

R₁ and R₂ independently represent hydrogen, (C₁₋₄) alkyl, benzyloxy, mono- and di-(C₁₋₃) alkylamino-(C₁₋₄) alkyl, (C₁₋₃)alkoxy-(C₁₋₄)alkyl, hydroxymethyl, hydroxy-(C₂₋₄)-alkyl, nitro or R₁ and R₂ taken together with two consecutive carbon atoms of the pyridine nucleus form a benzene ring unsubstituted or substituted by one or two methyl or ethyl groups; R₃ is a hydrogen atom or nil; with the proviso that, when A is A₁, -x- is nil and R₃ is a hydrogen atom; with the further proviso that, when A is A₂, -x- is a covalent chemical bond and R₃ is nil.

Also described herein is a compound as defined above, wherein A is A₁ or A₂ as above indicated, -x- is a covalent chemical bond or nil, R is hydrogen or acetyl, R₁ and R₂ independently represent hydrogen, (C₁₋₄)alkyl, benzyloxy, hydroxy-(C₂₋₄) alkyl, di-(C₁₋₃) alkylamino-(C₁₋₄) alkyl, nitro or R₁ and R₂ taken together with two consecutive carbon atoms of the pyridine nucleus form a benzene ring and R₃ is a hydrogen atom or nil; with the proviso that, when A is A₁, -x- is nil and R₃ is a hydrogen atom; with the further proviso that, when A is A₂, -x- is a covalent chemical bond and R₃ is nil.

Also described herein is a compound as defined above, wherein A is A₁ or A₂ as above indicated, -x- is a covalent chemical bond or nil, R is acetyl, R₁ and R₂ independently represent hydrogen, (C₁₋₄) alkyl or R₁ and R₂ taken together with two consecutive carbon atoms of the pyridine nucleus form a benzene ring and R₃ is a hydrogen atom or nil; with the proviso that, when A is A₁, -x- is nil and R₃ is a hydrogen atom; with the further proviso that, when A is A₂, -x- is a covalent chemical bond and R₃ is nil.

Also described herein is a compound as defined above, which is 4-deoxy-4′-methyl-pyrido[1′,2′-1,2]imidazo[5,4-c]rifamycin SV. Also described herein is a compound as defined above, which is 4-deoxy-pyrido[1′,2′:1,2]imidazo[5,4-c]rifamycin SV.

Also described herein is a compound as defined above, wherein A is as described above, -x- is a covalent chemical bond or nil; R is hydrogen or acetyl; R₁ and R₂ independently represent hydrogen, (C₁₋₄) alkyl, benzyloxy, mono- and di-(C₁₋₃)alkylamino(C₁₋₄)alkyl, (C₁₋₃)alkoxy-(C₁₋₄)alkyl, hydroxymethyl, hydroxy-(C₂₋₄)-alkyl, nitro or R₁ and R₂ taken together with two consecutive carbon atoms of the pyridine nucleus form a benzene ring unsubstituted or substituted by one or two methyl or ethyl groups; R₃ is a hydrogen atom or nil; with the proviso that, when A is A₁, -x- is nil and R₃ is a hydrogen atom; with the further proviso that, when A is A₂, -x- is a covalent chemical bond and R₃ is nil.

Rifaximin is a compound having the structure of formula II:

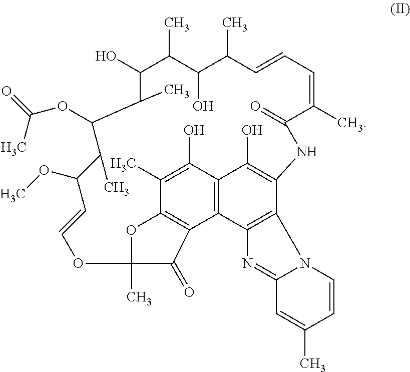

BD-Associated Sequences

A BD sequence can be initially identified by substantial nucleic acid and/or amino acid sequence homology to the BD sequences outlined herein. Such homology can be based upon the overall nucleic acid or amino acid sequence, and is generally determined as outlined below, using either homology programs or hybridization conditions. In one embodiment, BD sequences are those that are up-regulated in BDs; that is, the expression of these genes is higher in BD tissue as compared to normal tissue. “Up-regulation” as used herein means increased expression by about 50%, preferably about 100%, more preferably about 150% to about 200%, with up-regulation from 300% to 1000%.

In another embodiment, BD sequences are those that are down-regulated in BDs; that is, the expression of these genes is lower in BD tissue as compared to normal tissue of the same differentiation stage. “Down-regulation” as used herein means decreased expression by about 50%, preferably about 100%, more preferably about 150% to about 200%, with down-regulation from 300% to 1000% to no expression.

In yet another embodiment, BD sequences are those that have altered sequences but show either the same or an altered expression profile as compared to normal lymphoid tissue of the same differentiation stage. “Altered BD sequences” as used herein also refers, for example, to sequences that are truncated, contain insertions or contain point mutations.

In one embodiment, the BD sequences are transmembrane proteins. Transmembrane proteins are molecules that span the phospholipid bilayer of a cell. They may have an intracellular domain, an extracellular domain, or both. The intracellular domains of such proteins may have a number of functions including those already described for intracellular proteins. Proteins may also be intra-cellular, intra-nuclear, or secreted.

In general, the term “polypeptide” as used herein refers, for example, to both the full-length polypeptide encoded by the recited polynucleotide, the polypeptide encoded by the gene represented by the recited polynucleotide, as well as portions or fragments thereof. The present invention encompasses variants of the naturally occurring proteins, wherein such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant polypeptides have a sequence that has at least about 80%, at least about 81%, at least about 82%, at least about 83%, at least about 84%, at least about 85%, at least about 86%, at least about 87%, at least about 88%, at least about 89%, usually at least about 90%, at least 91%, at least 92%, at least 93%, at least 94%, at least 95%, at least 96%, at least 97%, at least 98% and more usually at least about 99% sequence identity with a differentially expressed polypeptide described herein.

Also within the scope of the invention are variants. Variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the protein (e.g., a functional domain and/or, where the polypeptide is a member of a protein family, a region associated with a consensus sequence).

Variants also include fragments of the polypeptides disclosed herein, particularly biologically active fragments and/or fragments corresponding to functional domains. Fragments of interest will typically be at least about 8 amino acids (aa) 10 aa, 15 aa, 20 aa, 25 aa, 30 aa, 35 aa, 40 aa, to at least about 45 aa in length, usually at least about 50 aa in length, at least about 75 aa, at least about 100 aa, at least about 125 aa, at least about 150 aa in length, at least about 200 aa, at least about 300 aa, at least about 400 aa and can be as long as 500 aa in length or longer, but will usually not exceed about 1000 aa in length, where the fragment will have a stretch of amino acids that is identical to a polypeptide encoded by a polynucleotide having a sequence of any one of the polynucleotide sequences provided herein, or a homolog thereof. The protein variants described herein are encoded by polynucleotides that are within the scope of the invention. The genetic code can be used to select the appropriate codons to construct the corresponding variants.

While altered expression of the polynucleotides associated with BD is observed, altered levels of expression of the polypeptides encoded by these polynucleotides may likely play a role in BDs.

Also included with the definition of BD protein in one embodiment are other BD proteins of the BD family, and BD proteins from other organisms, which are cloned and expressed as outlined below. Thus, probe or degenerate polymerase chain reaction (PCR) primer sequences may be used to find other related BD proteins from humans or other organisms. As will be appreciated by those in the art, particularly useful probe and/or PCR primer sequences include the unique areas of the BD nucleic acid sequence. As is generally known in the art, certain PCR primers are from about 15 to about 35 nucleotides in length, with from about 20 to about 30 being certain, and may contain inosine as needed. The conditions for the PCR reaction are well known in the art. In addition, as is outlined herein, BD proteins can be made that are longer than those encoded by the nucleic acids of the figures, for example, by the elucidation of additional sequences, the addition of epitope or purification tags, the addition of other fusion sequences, etc. BD proteins may also be identified as being encoded by BD nucleic acids. Thus, BD proteins are encoded by nucleic acids that will hybridize to the sequences of the sequence listings, or their complements, as outlined herein.

A BD sequence is initially identified by substantial nucleic acid and/or amino acid sequence homology to the BD sequences outlined herein. Such homology can be based upon the overall nucleic acid or amino acid sequence, and is generally determined as outlined below, using either homology programs or hybridization conditions.

As used herein, a nucleic acid is an “BD nucleic acid” if the overall homology of the nucleic acid sequence to one of the nucleic acids described herein preferably greater than about 75%, greater than about 80%, greater than about 85% or greater than 90%. In some embodiments the homology will be as high as about 93 to 95 or 98%. In one embodiment, the sequences that are used to determine sequence identity or similarity are selected from those of the nucleic acids described herein. In another embodiment, the sequences are naturally occurring allelic variants of the sequences of the nucleic acids described herein. In another embodiment, the sequences are sequence variants as further described herein.

Homology in this context means sequence similarity or identity. A comparison for homology purposes is to compare the sequence containing sequencing errors to the correct sequence. This homology will be determined using standard techniques known in the art, including, but not limited to, the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, PNAS USA 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12:387-395 (1984), preferably using the default settings, or by inspection. One example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters include a default gap weight of 3.00, a default gap length weight of 0.10, and weighted end gaps. Another example of a useful algorithm is the BLAST (Basic Local Alignment Search Tool) algorithm, described in Altschul et al., J. Mol. Biol. 215, 403-410, (1990) and Karlin et al., PNAS USA 90:5873-5787 (1993). A particularly useful BLAST program is the WU-BLAST-2 program which was obtained from Altschul et al., Methods in Enzymology, 266: 460-480 (1996); http://blast.wustl.edu/]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A percent amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored). Thus, “percent (%) nucleic acid sequence identity” is defined as the percentage of nucleotide residues in a candidate sequence that are identical with the nucleotide residues of the nucleic acids of Accession Nos.: NM_{—}033013; NM_{—}009803; NM_{—}022002; NM_{—}003889; CS618137; CS618135; or CS618133.

In another embodiment of the invention, polynucleotide compositions are provided that are capable of hybridizing under moderate to high stringency conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC (“saline sodium citrate”; 9 mM NaCl, 0.9 mM sodium citrate), 0.5% SDS, 1.0 mM EDTA (pH 8.0); hybridizing at 50-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60-65° C., or 65-70° C. Stringent conditions may also be achieved with the addition of destabilizing agents such as formamide.

In addition, the BD nucleic acid sequences of the invention are fragments of larger genes, e.g., they are nucleic acid segments. Alternatively, the BD nucleic acid sequences can serve as indicators of oncogene position, for example, the BD sequence may be an enhancer that activates a protooncogene. “Genes” in this context includes coding regions, non-coding regions, and mixtures of coding and non-coding regions. Accordingly, as will be appreciated by those in the art, using the sequences provided herein, additional sequences of the BD genes can be obtained, using techniques well known in the art for cloning either longer sequences or the full-length sequences; see Maniatis et al., and Ausubel, et al., supra, hereby expressly incorporated by reference. In general, this is done using PCR, for example, kinetic PCR.

Detection of BD Expression

Once the BD nucleic acid is identified, it can be cloned and, if necessary, its constituent parts recombined to form the entire BD nucleic acid. Once isolated from its natural source, e.g., contained within a plasmid or other vector or excised therefrom as a linear nucleic acid segment, the recombinant BD nucleic acid can be further used as a probe to identify and isolate other BD nucleic acids, for example additional coding regions. It can also be used as a “precursor” nucleic acid to make modified or variant BD nucleic acids and proteins. In one embodiment, once a BD gene is identified its nucleotide sequence is used to design probes specific for the BD gene.

The BD nucleic acids of the present invention are used in several ways. In a first embodiment, nucleic acid probes hybridizable to BD nucleic acids are made and attached to biochips to be used in screening and diagnostic methods, or for gene therapy and/or antisense applications. Alternatively, the BD nucleic acids that include coding regions of BD proteins can be put into expression vectors for the expression of BD proteins, again either for screening purposes or for administration to a subject.

Recent developments in DNA microarray technology make it possible to conduct a large scale assay of a plurality of target BD nucleic acid molecules on a single solid phase support. U.S. Pat. No. 5,837,832 (Chee et al.) and related patent applications describe immobilizing an array of oligonucleotide probes for hybridization and detection of specific nucleic acid sequences in a sample. Target polynucleotides of interest isolated from a tissue of interest are hybridized to the DNA chip and the specific sequences detected based on the target polynucleotides' preference and degree of hybridization at discrete probe locations. One use of arrays is in the analysis of differential gene expression, where the profile of expression of genes in different cells, often a cell of interest and a control cell, is compared and any differences in gene expression among the respective cells are identified. Such information is useful for the identification of the types of genes expressed in a particular cell or tissue type and diagnosis of BD conditions based on the expression profile. See U.S. Pat. No. 6,410,229 (Lockhart et al.). For example, use of a cDNA microarray to analyze gene expression patterns in human cancer is described by DeRisi, et al. (Nature Genetics 14:457-460 (1996)).

In certain embodiments, the probe can be a chimeric molecule; e.g., can comprise more than one type of base or sugar subunit, and/or the linkages can be of more than one type within the same primer. The probe can comprise a moiety to facilitate hybridization to its target sequence, as are known in the art, for example, intercalators and/or minor groove binders. Variations of the bases, sugars, and internucleoside backbone, as well as the presence of any pendant group on the probe, will be compatible with the ability of the probe to bind, in a sequence-specific fashion, with its target sequence. A large number of structural modifications, both known and to be developed, are possible within these bounds. Advantageously, the probes according to the present invention may have structural characteristics such that they allow the signal amplification, such structural characteristics being, for example, branched DNA probes as those described by Urdea et al. (Nucleic Acids Symp. Ser., 24:197-200 (1991)) or in the European Patent No. EP-0225,807. Moreover, synthetic methods for preparing the various heterocyclic bases, sugars, nucleosides and nucleotides that form the probe, and preparation of oligonucleotides of specific predetermined sequence, are well-developed and known in the art. One method for oligonucleotide synthesis incorporates the teaching of U.S. Pat. No. 5,419,966.

In one embodiment, BD nucleic acids encoding BD proteins are used to make a variety of expression vectors to express BD proteins which can then be used in screening assays, as described below. The expression vectors may be either self-replicating extrachromosomal vectors or vectors which integrate into a host genome. Generally, these expression vectors include transcriptional and translational regulatory nucleic acid operably linked to the nucleic acid encoding the BD protein. The term “control sequences” refers, for example, to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In one embodiment, the regulatory sequences include a promoter and transcriptional start and stop sequences. Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a prokaryotic host for cloning and amplification. In addition, in one embodiment, the expression vector contains a selectable marker gene to allow the selection of transformed host cells. Selection genes are well known in the art and will vary with the host cell used.

The BD proteins of the present invention may be produced, for example, by culturing a host cell transformed with an expression vector containing nucleic acid encoding a BD protein, under the appropriate conditions to induce or cause expression of the BD protein. The conditions appropriate for BD protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

The BD protein may also be made as a fusion protein, using techniques well known in the art.

In one embodiment, the BD nucleic acids, proteins and antibodies of the invention are labeled. By “labeled” herein is meant that a compound is either directly or indirectly labeled and may have at least one element which provides a detectable signal, for example, an isotope or chemical compound attached to enable the detection of the compound. In general, labels include, isotopic labels, which may be radioactive or heavy isotopes; magnetic labels; enzyme label, immune labels, which may be antibodies or antigens; and colored or fluorescent dyes. The labels may be incorporated into the BD nucleic acids, proteins and antibodies at any position. For example, the label should be capable of producing, either directly or indirectly, a detectable signal. The detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an enzyme, such as alkaline phosphatase, beta-galactosidase or horseradish peroxidase. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982). Other labels may include specific binding molecules. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In one embodiment, the binding of the candidate bioactive agent is determined through the use of competitive binding assays. In this embodiment, the competitor is a binding moiety known to bind to the target molecule (e.g., BD protein), such as an antibody, peptide, binding partner, ligand, etc. Under certain circumstances, there may be competitive binding as between the bioactive agent and the binding moiety, with the binding moiety displacing the bioactive agent.

BD Antigens and Antibodies Thereto

In one embodiment, the invention provides BD specific antibodies. In one embodiment, when the BD protein is to be used to generate antibodies, for example for immunotherapy, the BD protein should share at least one epitope or determinant with the full-length protein. By “epitope” or “determinant” herein is meant a portion of a protein that will generate and/or bind an antibody or T-cell receptor in the context of MHC. Thus, in most instances, antibodies made to a smaller BD protein will be able to bind to the full-length protein. In one embodiment, the epitope is unique; that is, antibodies generated to a unique epitope show little or no cross-reactivity. Any polypeptide sequence encoded by the BD polynucleotide sequences may be analyzed to determine certain regions of the polypeptide. Regions of high antigenicity are determined, for example, from data by DNASTAR analysis by choosing values that represent regions of the polypeptide that are likely to be exposed on the surface of the polypeptide in an environment in which antigen recognition may occur in the process of initiation of an immune response. For example, the amino acid sequence of a polypeptide encoded by a BD polynucleotide sequence may be analyzed using the default parameters of the DNASTAR computer algorithm (DNASTAR, Inc., Madison, Wis.; found on the world wide web at dnastar.com).

In one embodiment, the term “antibody” includes antibody fragments, as are known in the art, including Fab, Fab₂, single chain antibodies (Fv for example), chimeric antibodies, etc., either produced by the modification of whole antibodies or those synthesized de novo using recombinant DNA technologies. Methods of preparing polyclonal antibodies are known to the skilled artisan. Polyclonal antibodies can be raised in a mammal, for example, by one or more injections of an immunizing agent and, if desired, an adjuvant. The antibodies may, alternatively, be monoclonal antibodies. Monoclonal antibodies may be prepared using hybridoma methods, such as those described by Kohler and Milstein, Nature, 256:495 (1975). In one embodiment, the antibodies to BD are capable of reducing or eliminating the biological function of BD, as is described below. That is, the addition of anti-BD antibodies (either polyclonal or preferably monoclonal) to BD (or cells containing BD) may reduce or eliminate the BD activity. In one embodiment the antibodies to the BD proteins are humanized antibodies. “Humanized” antibodies refer to a molecule having an antigen binding site that is substantially derived from an immunoglobulin from a non-human species and the remaining immunoglobulin structure of the molecule based upon the structure and/or sequence of a human immunoglobulin. Specific antibodies, either polyclonal or monoclonal, to the BD proteins can be produced by any suitable method known in the art as discussed above. For example, murine or human monoclonal antibodies can be produced by hybridoma technology or, alternatively, the BD proteins, or an immunologically active fragment thereof, or an anti-idiotypic antibody, or fragment thereof can be administered to an animal to elicit the production of antibodies capable of recognizing and binding to the BD proteins. Such antibodies can be from any class of antibodies including, but not limited to IgG, IgA, IgM, IgD, and IgE or in the case of avian species, IgY and from any subclass of antibodies.

In another certain embodiment, the BD protein to which antibodies are raised is a transmembrane protein. Without being bound by theory, antibodies used for treatment, bind the extracellular domain of the BD protein and prevent it from binding to other proteins, such as circulating ligands or cell-associated molecules. The antibody may cause down-regulation of the transmembrane BD protein. As will be appreciated by one of ordinary skill in the art, the antibody may be a competitive, non-competitive or uncompetitive inhibitor of protein binding to the extracellular domain of the BD protein. The antibody is also an antagonist of the BD protein. Further, the antibody prevents activation of the transmembrane BD protein. In one aspect, when the antibody prevents the binding of other molecules to the BD protein, the antibody prevents growth of the cell. The antibody may also sensitize the cell to cytotoxic agents, including, but not limited to TNF-.alpha., TNF-.beta., IL-1, INF-.gamma. and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity. Thus, BDs may be treated by administering to a subject antibodies directed against the transmembrane BD protein.

In another certain embodiment, the antibody is conjugated to a therapeutic moiety. In one aspect the therapeutic moiety is a small molecule that modulates the activity of the BD protein. In another aspect the therapeutic moiety modulates the activity of molecules associated with or in close proximity to the BD protein. The therapeutic moiety may inhibit enzymatic activity such as protease or protein kinase activity associated with BD.

In another certain embodiment, the BD protein against which the antibodies are raised is an intracellular protein. In this case, the antibody may be conjugated to a protein that facilitates entry into the cell. In one case, the antibody enters the cell by endocytosis. In another embodiment, a nucleic acid encoding the antibody is administered to the individual or cell. Moreover, wherein the BD protein can be targeted within a cell, e.g., the nucleus, an antibody thereto contains a signal for that target localization, e.g., a nuclear localization signal.

The BD antibodies of the invention specifically bind to BD proteins. By “specifically bind” herein is meant that the antibodies bind to the protein with a binding constant in the range of 10⁴-10⁻⁶ M, with one range being 10⁻⁷-10⁻⁹ M. In one embodiment, the BD protein is purified or isolated after expression. BD proteins may be isolated or purified in a variety of ways known to those skilled in the art depending on what other components are present in the sample.

Production of antibodies described herein include, for example, methods for the production of antibodies capable of specifically recognizing one or more epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of a PXR gene in a biological sample, or, alternatively, as a method for the inhibition of abnormal PXR gene activity. Thus, such antibodies may be utilized as part of disease treatment methods, and/or may be used as part of diagnostic techniques whereby subjects may be tested for abnormal levels of PXR gene proteins, or for the presence of abnormal forms of such proteins.

For the production of antibodies, various host animals may be immunized by injection with the PXR gene, its expression product or a portion thereof. Such host animals may include but are not limited to rabbits, mice, rats, goats and chickens, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as PXR gene product, or an antigenic functional derivative thereof. For the production of polyclonal antibodies, host animals such as those described above, may be immunized by injection with gene product supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to the hybridoma technique of Kohler and Milstein, Nature, 256:495-7 (1975); and U.S. Pat. No. 4,376,110), the human B-cell hybridoma technique (Kosbor, et al., Immunology Today, 4:72 (1983); Cote, et al., Proc. Natl. Acad. Sci. USA, 80:2026-30 (1983)), and the EBV-hybridoma technique (Cole, et al., in Monoclonal Antibodies And BD Therapy, Alan R. Liss, Inc., New York, pp. 77-96 (1985)). Such antibodies may be of any immunoglobulin class including IgG, IgM, IgE, IgA, IgD and any subclass thereof. The hybridoma producing the mAb of this disclosure may be cultivated in vitro or in vivo. Production of high titers of mAbs in vivo makes this the presently certain method of production.

In addition, techniques developed for the production of “chimeric antibodies” (Morrison, et al., Proc. Natl. Acad. Sci., 81:6851-6855 (1984); Takeda, et al., Nature, 314:452-54 (1985)) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, Science 242:423-26 (1988); Huston, et al., Proc. Natl. Acad. Sci. USA, 85:5879-83 (1988); and Ward, et al., Nature, 334:544-46 (1989)) can be adapted to produce gene-single chain antibodies. Single chain antibodies are typically formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)₂ fragments that can be produced by pepsin digestion of the antibody molecule and the Fab fragments that can be generated by reducing the disulfide bridges of the F(ab′)₂ fragments. Alternatively, Fab expression libraries may be constructed (Huse, et al., Science, 246:1275-81 (1989)) to allow rapid and easy identification of monoclonal Fab fragments with the desired specificity.

Detection of BD Phenotype

Once expressed and purified, if necessary, the BD proteins and nucleic acids are useful in a number of applications. In one aspect, the expression levels of genes are determined for different cellular states in the BD phenotype; that is, the expression levels of genes in normal tissue and in BD tissue are evaluated to provide expression profiles. An expression profile of a particular cell state or point of development is essentially a “fingerprint” of the state; while two states may have any particular gene similarly expressed, the evaluation of a number of genes simultaneously allows the generation of a gene expression profile that is unique to the state of the cell. By comparing expression profiles of cells in different states, information regarding which genes (including both up- and down-regulation of genes) in each of these states is obtained. Then, diagnosis may be done or confirmed, for example, does tissue from a particular subject have the gene expression profile of normal or BD tissue.

In one embodiment, any of the three classes of proteins as described herein (secreted, transmembrane or intracellular proteins) are used in diagnostic assays. The BD proteins, antibodies, nucleic acids, modified proteins and cells containing BD sequences are used in diagnostic assays. This can be done on a subject gene or corresponding polypeptide level, or as sets of assays.

In another certain method, antibodies to the BD protein find use in in situ imaging techniques. In this method cells are contacted with from one to many antibodies to the BD protein(s). Following washing to remove non-specific antibody binding, the presence of the antibody or antibodies is detected. In one embodiment the antibody is detected by incubating with a secondary antibody that contains a detectable label. In another method the primary antibody to the BD protein(s) contains a detectable label. In another certain embodiment each one of multiple primary antibodies contains a distinct and detectable label. This method finds particular use in simultaneous screening for a plurality of BD proteins. As will be appreciated by one of ordinary skill in the art, numerous other histological imaging techniques are useful in the invention.

It is understood that when comparing the expression fingerprints between a subject and a standard, the skilled artisan can make a diagnosis as well as a prognosis. It is further understood that the genes that indicate diagnosis may differ from those that indicate prognosis.

Screening for Candidate Compositions

In one embodiment, any of the BD sequences as described herein are used in drug screening assays. The BD proteins, antibodies, nucleic acids, modified proteins and cells containing BD sequences are used in drug screening assays or by evaluating the effect of drug candidates on a “gene expression profile” or expression profile of polypeptides. In one embodiment, the expression profiles are used, preferably in conjunction with high throughput screening techniques to allow monitoring for expression profile genes after treatment with a candidate agent, Zlokarnik, et al., Science 279, 84-8 (1998), Heid, et al., Genome Res., 6:986-994 (1996).

In another embodiment, the BD proteins, antibodies, nucleic acids, modified proteins and cells containing the native or modified BD proteins are used in screening assays. That is, the present invention provides novel methods for screening for compositions that modulate the BD phenotype. As above, this can be done by screening for modulators of gene expression or for modulators of protein activity. Similarly, this may be done on a subject gene or protein level or by evaluating the effect of drug candidates on a “gene expression profile”. In one embodiment, the expression profiles are used, preferably in conjunction with high throughput screening techniques to allow monitoring for expression profile genes after treatment with a candidate agent, see Zlokarnik, supra.

Having identified the BD genes herein, a variety of assays to evaluate the effects of agents on gene expression may be executed. In one embodiment, assays may be run on a subject gene or protein level. That is, having identified a particular gene as aberrantly regulated in BD, candidate bioactive agents may be screened to modulate the gene's regulation. “Modulation” thus includes both an increase and a decrease in gene expression or activity. The certain amount of modulation will depend on the original change of the gene expression in normal versus BD tissue, with changes of at least 10%, preferably 50%, more preferably 100-300%, and in some embodiments 300-1000% or greater. Thus, if a gene exhibits a 4 fold increase in BD compared to normal tissue, a decrease of about four fold is desired; a 10 fold decrease in BD compared to normal tissue gives a 10 fold increase in expression for a candidate agent is desired, etc. Alternatively, where the BD sequence has been altered but shows the same expression profile or an altered expression profile, the protein will be detected as outlined herein.

As will be appreciated by those in the art, this may be done by evaluation at either the gene or the protein level; that is, the amount of gene expression may be monitored using nucleic acid probes and the quantification of gene expression levels, or, alternatively, the level of the gene product itself can be monitored, for example through the use of antibodies to the BD protein and standard immunoassays. Alternatively, binding and bioactivity assays with the protein may be done as outlined below.

In one embodiment, gene expression monitoring is done and a number of genes, e.g., an expression profile, is monitored simultaneously, although multiple protein expression monitoring can be done as well.

Generally, in one embodiment, a candidate bioactive agent is added to the cells prior to analysis. Moreover, screens are provided to identify a candidate bioactive agent that modulates a particular type of BD, modulates BD proteins, binds to a BD protein, or interferes between the binding of a BD protein and an antibody.

The term “candidate bioactive agent,” “candidate agent,” “candidate composition,” or “drug candidate” or grammatical equivalents as used herein describes any molecule, e.g., protein, oligopeptide, small organic or inorganic molecule, polysaccharide, polynucleotide, etc., to be tested for bioactive agents that are capable of directly or indirectly altering either the BD phenotype, binding to and/or modulating the bioactivity of a BD protein, or the expression of a BD sequence, including both nucleic acid sequences and protein sequences. In a particularly certain embodiment, the candidate agent suppresses a BD phenotype, for example to a normal tissue fingerprint. Similarly, the candidate agent preferably suppresses a severe BD phenotype. Generally a plurality of assay mixtures are run in parallel with different agent concentrations to obtain a differential response to the various concentrations. Typically, one of these concentrations serves as a negative control, e.g., at zero concentration or below the level of detection.

In one aspect, a candidate agent will neutralize the effect of a BD protein. By “neutralize” is meant that activity of a protein is either inhibited or counter acted against so as to have substantially no effect on a cell.

Candidate agents encompass numerous chemical classes, though typically they are organic or inorganic molecules, preferably small organic compounds having a molecular weight of more than 100 and less than about 2,500 Daltons. Small molecules, may be, for example, less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, or amidification to produce structural analogs. In one embodiment, the candidate bioactive agents are proteins. In one embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of prokaryotic and eukaryotic proteins may be made for screening in the methods of the invention. In some embodiments, libraries are made of, for example, bacterial, fungal, viral, and mammalian proteins.

In another embodiment, the candidate bioactive agents are peptides of from about 5 to about 30 amino acids, from about 5 to about 20 amino acids or from about 7 to about 15 amino acids. The peptides may be digests of naturally occurring proteins as is outlined above, random peptides, or “biased” random peptides.

In one embodiment, the candidate bioactive agents are nucleic acids. As described generally for proteins, nucleic acid candidate bioactive agents may be naturally occurring nucleic acids, random nucleic acids, or “biased” random nucleic acids. In another embodiment, the candidate bioactive agents are organic chemical moieties, a wide variety of which are available in the literature.

During or after an assay is run, the data are analyzed to determine the expression levels, and changes in expression levels as between states, of individual genes, forming a gene expression profile.

In one embodiment, as for the diagnosis and prognosis applications, having identified the differentially expressed gene(s) or mutated gene(s) in any one state, screens can be run to test for alteration of the expression of the BD genes individually. That is, screening for modulation of regulation of expression of a single gene can be done. Thus, for example, in the case of target genes whose presence or absence is unique between two states, screening is done for modulators of the target gene expression. In one embodiment, the candidate bioactive agent is labeled. Either the candidate bioactive agent, or the competitor, or both, is added first to the protein for a time sufficient to allow binding, if present. Incubations may be performed at any temperature which facilitates optimal activity, typically between 4 and 40° C. Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In one embodiment, the competitor is added first, followed by the candidate bioactive agent. Displacement of the competitor is an indication that the candidate bioactive agent is binding to the BD protein and thus is capable of binding to, and potentially modulating, the activity of the BD protein. In this embodiment, either component can be labeled. Thus, for example, if the competitor is labeled, the presence of label in the wash solution indicates displacement by the agent. Alternatively, if the candidate bioactive agent is labeled, the presence of the label on the support indicates displacement.

In an alternative embodiment, the candidate bioactive agent is added first, with incubation and washing, followed by the competitor. The absence of binding by the competitor may indicate that the bioactive agent is bound to the BD protein with a higher affinity. Thus, if the candidate bioactive agent is labeled, the presence of the label on the support, coupled with a lack of competitor binding, may indicate that the candidate agent is capable of binding to the BD protein.

In one embodiment, the methods comprise differential screening to identity bioactive agents that are capable of modulating the activity of the BD proteins. In this embodiment, the methods comprise combining a BD protein and a competitor in a first sample. A second sample comprises a candidate bioactive agent, a BD protein and a competitor. The binding of the competitor is determined for both samples, and a change, or difference in binding between the two samples indicates the presence of an agent capable of binding to the BD protein and potentially modulating its activity. That is, if the binding of the competitor is different in the second sample relative to the first sample, the agent is capable of binding to the BD protein.

Screening for agents that modulate the activity of BD proteins may also be done. In one embodiment, methods for screening for a bioactive agent capable of modulating the activity of BD proteins comprise the steps of adding a candidate bioactive agent to a sample of BD proteins, as above, and determining an alteration in the biological activity of BD proteins. “Modulating the activity of a BD protein” includes an increase in activity, a decrease in activity, or a change in the type or kind of activity present. Thus, in this embodiment, the candidate agent should both bind to BD proteins (although this may not be necessary), and alter its biological or biochemical activity as defined herein. The methods include both in vitro screening methods, as are generally outlined above, and in vivo screening of cells for alterations in the presence, distribution, activity or amount of BD proteins.

Thus, in this embodiment, the methods comprise combining a BD sample and a candidate bioactive agent, and evaluating the effect on BD activity. By “BD activity” or grammatical equivalents herein is meant one of the BD protein's biological activities, including, but not limited to, its role in BD.

In one embodiment, the activity of the BD protein is increased; in another embodiment, the activity of the BD protein is decreased. Thus, bioactive agents that are antagonists are in some embodiments, and bioactive agents that are agonists in other embodiments.

Applications

In one embodiment, a method of inhibiting BD is provided. In another embodiment, a method of amelorating BD is provided. In a further embodiment, methods of treating cells or individuals with BD are provided.

The method comprises administration of a BD inhibitor. In particular embodiments, the BD inhibitor is an antisense molecule, a pharmaceutical composition, a therapeutic agent or small molecule, or a monoclonal, polyclonal, chimeric or humanized antibody. In particular embodiments, a therapeutic agent is coupled with an antibody, preferable a monoclonal antibody. In particular embodiments, the BD inhibitor is a PXR activator. For example, a rifamycin analog. Rifamycin analogs include, for example, rifaximin and rifamycin.

In other embodiments, methods for detection or diagnosis of BD cells in a subject are provided. In particular embodiments, the diagnostic/detection agent is a small molecule that preferentially binds to a BDP according to the invention. In one embodiment, the diagnostic/detection agent is an antibody, e.g., a monoclonal antibody, optionally linked to a detectable agent.

In other embodiments of the invention, animal models and transgenic animals are provided, which find use in generating animal models of BD.

Antisense, Ribozymes, and Antibodies

Other agents that may be used as therapeutics include the PXR gene, its expression product(s) and functional fragments thereof. Additionally, agents that reduce or inhibit mutant PXR gene activity may be used to ameliorate disease symptoms. Such agents include antisense, ribozyme, and triple helix molecules. Techniques for the production and use of such molecules are well known to those of skill in the art.

Ribozymes are enzymatic RNA molecules capable of catalyzing the specific cleavage of RNA. The mechanism of ribozyme action involves sequence-specific hybridization of the ribozyme molecule to complementary target RNA, followed by an endonucleolytic cleavage. The composition of ribozyme molecules must include one or more sequences complementary to the PXR gene mRNA, and must include the well known catalytic sequence responsible for mRNA cleavage. For this sequence, see U.S. Pat. No. 5,093,246, which is incorporated by reference herein in its entirety. As such within the scope of the disclosure are engineered hammerhead motif ribozyme molecules that specifically and efficiently catalyze endonucleolytic cleavage of RNA sequences encoding PXR gene proteins.

Specific ribozyme cleavage sites within any potential RNA target are initially identified by scanning the molecule of interest for ribozyme cleavage sites that include the following sequences, GUA, GUU and GUC. Once identified, short RNA sequences of between 15 and 20 ribonucleotides corresponding to the region of the PXR gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

It is possible that the antisense, ribozyme, and/or triple helix molecules described herein may reduce or inhibit the transcription (triple helix) and/or translation (antisense, ribozyme) of mRNA produced by both normal and mutant PXR gene alleles. In order to ensure that substantially normal levels of PXR gene activity are maintained, nucleic acid molecules that encode and express PXR gene polypeptides exhibiting normal activity may be introduced into cells that do not contain sequences susceptible to whatever antisense, ribozyme, or triple helix treatments are being utilized. Alternatively, it may be preferable to coadminister normal PXR gene protein into the cell or tissue in order to maintain the requisite level of cellular or tissue PXR gene activity.

Anti-sense RNA and DNA, ribozyme, and triple helix molecules of the disclosure may be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone.

Antibodies that are both specific for PXR gene protein, and in particular, mutant gene protein, and interfere with its activity may be used to inhibit mutant PXR gene function. Such antibodies may be generated against the proteins themselves or against peptides corresponding to portions of the proteins using standard techniques known in the art and as also described herein. Such antibodies include but are not limited to polyclonal, monoclonal, Fab fragments, single chain antibodies, chimeric antibodies, etc.

In instances where the PXR gene protein is intracellular and whole antibodies are used, internalizing antibodies may be certain. However, lipofectin liposomes may be used to deliver the antibody or a fragment of the Fab region that binds to the PXR gene epitope into cells. Where fragments of the antibody are used, the smallest inhibitory fragment that binds to the target or expanded target protein's binding domain is certain. For example, peptides having an amino acid sequence corresponding to the domain of the variable region of the antibody that binds to the PXR gene protein may be used. Such peptides may be synthesized chemically or produced via recombinant DNA technology using methods well known in the art (see, e.g., Creighton, Proteins: Structures and Molecular Principles (1984) W. H. Freeman, New York 1983, supra; and Sambrook, et al., 1989, supra). Alternatively, single chain neutralizing antibodies that bind to intracellular PXR gene epitopes may also be administered. Such single chain antibodies may be administered, for example, by expressing nucleotide sequences encoding single-chain antibodies within the target cell population by utilizing, for example, techniques such as those described in Marasco, et al., Proc. Natl. Acad. Sci. USA, 90:7889-93 (1993).

RNA sequences encoding PXR gene protein may be directly administered to a subject exhibiting disease symptoms, at a concentration sufficient to produce a level of PXR gene protein such that disease symptoms are ameliorated. Subjects may be treated by gene replacement therapy. One or more copies of a normal PXR gene, or a portion of the gene that directs the production of a normal PXR gene protein with PXR gene function, may be inserted into cells using vectors that include, but are not limited to adenovirus, adeno-associated virus, and retrovirus vectors, in addition to other particles that introduce DNA into cells, such as liposomes. Additionally, techniques such as those described above may be utilized for the introduction of normal PXR gene sequences into human cells.

Cells, preferably, autologous cells, containing normal PXR gene expressing gene sequences may then be introduced or reintroduced into the subject at positions that allow for the amelioration of disease symptoms.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the PXR gene nucleotide sequence of interest, are certain.

In one embodiment, the BD inhibitor or activator is an antisense molecule, e.g., a PXR activator. Antisense molecules as used herein include antisense or sense oligonucleotides comprising a single-stranded nucleic acid sequence (either RNA or DNA) capable of binding to target mRNA (sense) or DNA (antisense) sequences for BD molecules. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, for example, Stein and Cohen, Cancer Res. 48:2659, (1988) and van der Krol et al., BioTechniques 6:958, (1988).

Antisense molecules can be modified or unmodified RNA, DNA, or mixed polymer oligonucleotides. These molecules function by specifically binding to matching sequences resulting in inhibition of peptide synthesis (Wu-Pong, November 1994, BioPharm, 20-33) either by steric blocking or by activating an RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190). In addition, binding of single stranded DNA to RNA can result in nuclease-mediated degradation of the heteroduplex (Wu-Pong, supra). Backbone modified DNA chemistry which have thus far been shown to act as substrates for RNase H are phosphorothioates, phosphorodithioates, borontrifluoridates, and 2′-arabino and 21-fluoro arabino-containing oligonucleotides.

RNA interference refers, for example, to the process of sequence-specific post transcriptional gene silencing in animals mediated by short interfering RNAs (siRNA) (Fire et al., Nature, 391, 806 (1998)). The corresponding process in plants is referred to as post transcriptional gene silencing or RNA silencing and is also referred to as quelling in fungi. The presence of dsRNA in cells triggers the RNAi response though a mechanism that has yet to be fully characterized. This mechanism appears to be different from the interferon response that results from dsRNA mediated activation of protein kinase PKR and 2′,5′-oligoadenylate synthetase resulting in non-specific cleavage of mRNA by ribonuclease L. (reviewed in Sharp, P. A., RNA interference—2001, Genes & Development 15:485-490 (2001)).

Provided herein are expression systems comprising an isolated nucleic acid molecule comprising a sequence capable of specifically hybridizing to the BD sequences. In an embodiment, the nucleic acid molecule is capable of inhibiting the expression of the BD protein. A method of inhibiting expression of BD inside a cell by a vector-directed expression of a short RNA which short RNA can fold in itself and create a double strand RNA having BD mRNA sequence identity and able to trigger posttranscriptional gene silencing, or RNA interference (RNAi), of the BD gene inside the cell. In another method a short double strand RNA having BD mRNA sequence identity is delivered inside the cell to trigger posttranscriptional gene silencing, or RNAi, of the BD gene. In various embodiments, the nucleic acid molecule is at least a 7 mer, at least a 10 mer, or at least a 20 mer. In a further embodiment, the sequence is unique.

Pharmaceutical Compositions

Pharmaceutical compositions provided herein include as active agent, the small molecules polypeptides, polynucleotides, antisense oligonucleotides, or antibodies disclosed herein in a therapeutically effective amount. An “effective amount” is an amount sufficient to effect beneficial or desired results, including clinical results. An effective amount can be administered in one or more administrations. For purposes of this invention, an effective amount of an adenoviral vector is an amount that is sufficient to palliate, ameliorate, stabilize, reverse, slow or delay the progression of the disease state.

The compositions can be used to treat BD. The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, e.g., arresting its development; or (c) relieving the disease symptom, e.g., causing regression of the disease or symptom.

A “subject” for the purposes of the present invention includes both humans and other animals, particularly mammals, and organisms. Thus the methods are applicable to both human therapy and veterinary applications. In the certain embodiment the subject is a mammal, and in the most certain embodiment the subject is human.

The term “therapeutically effective amount” as used herein refers, for example, to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. The effective amount for a given situation is determined by routine experimentation and is within the judgment of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg or about 0.1 mg/kg to about 100 mg/kg of the compositions of the present invention in the individual to which it is administered.

A pharmaceutical composition can also contain a pharmaceutically acceptable carrier. The term “pharmaceutically acceptable carrier” refers, for example, to a carrier for administration of a therapeutic agent, such as antibodies or a polypeptide, genes, and other therapeutic agents. The term refers, for example, to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylacetic acids, polyglycolic acids, polymeric amino acids, amino acid copolymers, and inactive virus particles. Such carriers are well known to those of ordinary skill in the art. Pharmaceutically acceptable carriers in therapeutic compositions can include liquids such as water, saline, glycerol and ethanol. Auxiliary substances, such as wetting or emulsifying agents, pH buffering substances, and the like, can also be present in such vehicles. Typically, the therapeutic compositions are prepared as injectables, either as liquid solutions or suspensions; solid forms suitable for solution in, or suspension in, liquid vehicles prior to injection can also be prepared. Liposomes are included within the definition of a pharmaceutically acceptable carrier. Pharmaceutically acceptable salts can also be present in the pharmaceutical composition, e.g., mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington: The Science and Practice of Pharmacy (1995) Alfonso Gennaro, Lippincott, Williams, & Wilkins.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

The pharmaceutical compositions of the present invention comprise a BD protein in a form suitable for administration to a subject. In the certain embodiment, the pharmaceutical compositions are in a water soluble form, such as being present as pharmaceutically acceptable salts, which is meant to include both acid and base addition salts. “Pharmaceutically acceptable acid addition salt” refers, for example, to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly certain are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The therapeutic polynucleotides and polypeptides of the present invention can be delivered using gene delivery vehicles. The gene delivery vehicle can be of viral or non-viral origin (see generally, Jolly, B D Gene Therapy (1994) 1:51; Kimura, Human Gene Therapy 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

The administration of the BD proteins and modulators of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the BD proteins and modulators may be directly applied as a solution or spray.

In one embodiment, methods of modulating BD gene activity in cells or organisms are provided. In one embodiment, the methods comprise administering to a cell an anti-BD antibody that reduces or eliminates the biological activity of an endogenous BD protein. Alternatively, the methods comprise administering to a cell or organism a recombinant nucleic acid encoding a BD protein. As will be appreciated by those in the art, this may be accomplished in any number of ways. In one embodiment, for example when the BD sequence is down-regulated in BD, the activity of the BD gene product is increased by increasing the amount of BD expression in the cell, for example by overexpressing the endogenous BD gene or by administering a gene encoding the BD sequence, using known gene-therapy techniques. In one embodiment, the gene therapy techniques include the incorporation of the exogenous gene using enhanced homologous recombination (EHR), for example as described in PCT/US93/03868, hereby incorporated by reference in its entirety. Alternatively, for example when the BD sequence is up-regulated in BD, the activity of the endogenous BD gene is decreased, for example by the administration of a BD antisense nucleic acid.

The detection methods can be provided as part of a kit. Thus, the invention further provides kits for detecting the presence and/or a level of a polynucleotide that is differentially expressed in a BD cell (e.g., by detection of an mRNA encoded by the differentially expressed gene of interest), and/or a polypeptide encoded thereby, in a biological sample. Procedures using these kits can be performed by clinical laboratories, experimental laboratories, medical practitioners, or private individuals. The kits of the invention for detecting a polypeptide encoded by a polynucleotide that is differentially expressed in a BD cell may comprise a moiety that specifically binds the polypeptide, which may be an antibody that binds the polypeptide or fragment thereof. The kits of the invention used for detecting a polynucleotide that is differentially expressed in a BD cell may comprise a moiety that specifically hybridizes to such a polynucleotide. The kit may optionally provide additional components that are useful in the procedure, including, but not limited to, buffers, developing reagents, labels, reacting surfaces, means for detection, control samples, standards, instructions, and interpretive information. Accordingly, the present invention provides kits for detecting prostate BD comprising at least one of polynucleotides having the sequence as shown in NCBI Accession Nos.: NM_{—}033013; NM_{—}009803; NM_{—}022002; NM_{—}003889; CS618137; CS618135; CS618133 or fragments thereof.

The identified compounds that inhibit target mutant gene expression, synthesis and/or activity can be administered to a subject at therapeutically effective doses to treat or ameliorate the disease. A therapeutically effective dose refers, for example, to that amount of the compound sufficient to result in amelioration of symptoms of the disease.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are certain. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound that achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present disclosure may be formulated in conventional manner using one or more physiologically acceptable carriers or excipients. Thus, the compounds and their physiologically acceptable salts and solvates may be formulated for administration by inhalation or insufflation (either through the mouth or the nose) or oral, buccal, parenteral, topical, subcutaneous, intraperitoneal, intraveneous, intrapleural, intraoccular, intraarterial, or rectal administration. It is also contemplated that pharmaceutical compositions may be administered with other products that potentiate the activity of the compound and optionally, may include other therapeutic ingredients.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound. For buccal administration the compositions may take the form of tablets or lozenges formulated in conventional manner.

For administration by inhalation, the compounds for use according to the present disclosure are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. Oral ingestion is possibly the easiest method of taking any medication. Such a route of administration, is generally simple and straightforward and is frequently the least inconvenient or unpleasant route of administration from the subject's point of view. However, this involves passing the material through the stomach, which is a hostile environment for many materials, including proteins and other biologically active compositions. As the acidic, hydrolytic and proteolytic environment of the stomach has evolved efficiently to digest proteinaceous materials into amino acids and oligopeptides for subsequent anabolism, it is hardly surprising that very little or any of a wide variety of biologically active proteinaceous material, if simply taken orally, would survive its passage through the stomach to be taken up by the body in the small intestine. The result, is that many proteinaceous medicaments must be taken in through another method, such as parenterally, often by subcutaneous, intramuscular or intravenous injection.

Pharmaceutical compositions may also include various buffers (e.g., Tris, acetate, phosphate), solubilizers (e.g., Tween, Polysorbate), carriers such as human serum albumin, preservatives (thimerosol, benzyl alcohol) and anti-oxidants such as ascorbic acid in order to stabilize pharmaceutical activity. The stabilizing agent may be a detergent, such as tween-20, tween-80, NP-40 or Triton X-100. EBP may also be incorporated into particulate preparations of polymeric compounds for controlled delivery to a subject over an extended period of time. A more extensive survey of components in pharmaceutical compositions is found in Remington's Pharmaceutical Sciences, 18th ed., A. R. Gennaro, ed., Mack Publishing, Easton, Pa. (1990).

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Pregnane X Receptors (PXRs)

Cells useful in the present invention include eukaryotic and prokaryotic cells, including, but not limited to, bacterial cells, yeast cells, fungal cells, insect cells, nematode cells, plant cells, and animal cells. Suitable animal cells include, but are not limited to, BDK cells, HeLa cells, COS cells, U20S cells, CHO-KI cells, and various primary mammalian cells.

Cells useful in the present invention include those that express PXR, unknown PXRs, a modified PXR, and combinations thereof. A cell that expresses a PXR is one that contains the PXR as a functional receptor in its cell membrane. The cells may naturally express the PXRs, may be genetically engineered to express the PXRs at varying levels of expression, or may be genetically engineered to inducibly express the PXRs. As one skilled in the art readily would understand, the cells may be genetically engineered to express PXRs by molecular biological techniques standard in the genetic engineering art.

In addition, cells useful in the present invention may stably or transiently express the PXRs used in the methods described herein. Methods of expressing genes using non-mammalian viruses (e.g., baculoviruses) described in U.S. Pat. Nos. 4,745,051; 4,879,236; 5,348,886; 5,731,182; 5,871,986; 6,281,009; and 6,238,914; may be used in the present methods. The entire contents of U.S. Pat. Nos. 4,745,051; 4,879,236; 5,348,886; 5,731,182; 5,871,986; 6,281,009; and 6,238,914 are hereby incorporated by reference herein in their entirety. Methods of detection that may be used with the methods of the present invention are also described in U.S. patent application Ser. No. 10/095,620, U.S. Pat. No. 5,891,646 and U.S. Pat. No. 6,110,693, the contents of which are hereby incorporated by reference herein in their entirety.

Animal Models and Transgenic Animals

In another embodiment BD genes find use in generating animal models of BDs. As is appreciated by one of ordinary skill in the art, when the BD gene identified is repressed or diminished in BD tissue, gene therapy technology wherein antisense RNA directed to the BD gene will also diminish or repress expression of the gene. An animal generated as such serves as an animal model of BD that finds use in screening bioactive drug candidates. Similarly, gene knockout technology, for example as a result of homologous recombination with an appropriate gene targeting vector, will result in the absence of the BD protein. When desired, tissue-specific expression or knockout of the BD protein may be necessary.

It is also possible that the BD protein is overexpressed in BD. As such, transgenic animals can be generated that overexpress the BD protein. Depending on the desired expression level, promoters of various strengths can be employed to express the transgene. Also, the number of copies of the integrated transgene can be determined and compared for a determination of the expression level of the transgene. Animals generated by such methods find use as animal models of BD and are additionally useful in screening for bioactive molecules to treat BD.

Generation of Targeting Construct

The targeting construct of the present disclosure may be produced using standard methods known in the art. (see, e.g., Sambrook, et al., 1989, Molecular Cloning: A Laboratory Manual, Second Edition, Cold Spring Harbor Laboratory Press, Cold Spring Harbor, N.Y.; E. N. Glover (eds.), 1985, DNA Cloning: A Practical Approach, Volumes I and II; M. J. Gait (ed.), 1984, Oligonucleotide Synthesis; B. D. Hames & S. J. Higgins (eds.), 1985, Nucleic Acid Hybridization; B. D. Hames & S. J. Higgins (eds.), 1984, Transcription and Translation; R. I. Freshney (ed.), 1986, Animal Cell Culture; Immobilized Cells and Enzymes, IRL Press, 1986; B. Perbal, 1984, A Practical Guide To Molecular Cloning; F. M. Ausubel et al., 1994, Current Protocols in Molecular Biology, John Wiley & Sons, Inc.). For example, the targeting construct may be prepared in accordance with conventional ways, where sequences may be synthesized, isolated from natural sources, manipulated, cloned, ligated, subjected to in vitro mutagenesis, primer repair, or the like. At various stages, the joined sequences may be cloned, and analyzed by restriction analysis, sequencing, or the like.

The targeting DNA can be constructed using techniques well known in the art. For example, the targeting DNA may be produced by chemical synthesis of oligonucleotides, nick-translation of a double-stranded DNA template, polymerase chain-reaction amplification of a sequence (or ligase chain reaction amplification), purification of prokaryotic or target cloning vectors harboring a sequence of interest (e.g., a cloned cDNA or genomic DNA, synthetic DNA or from any of the aforementioned combination) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. Moreover, the length of homology may be selected using known methods in the art. For example, selection may be based on the sequence composition and complexity of the predetermined endogenous target DNA sequence(s).

In one embodiment of the present disclosure, the targeting construct is prepared directly from a plasmid genomic library using the methods described in U.S. Pat. No. 6,815,185 issued Nov. 9, 2004, which is based on U.S. patent application Ser. No. 09/885,816, filed Jun. 19, 2001, which is a continuation of U.S. application Ser. No. 09/193,834, filed Nov. 17, 1998, now abandoned, which claims priority to provisional application No. 60/084,949, filed on May 11, 1998, and provisional application No. 60/084,194; and U.S. patent application Ser. No. 08/971,310, filed Nov. 17, 1997, which was converted to provisional application No.: 60/084,194; the disclosure of which is incorporated herein in its entirety. Generally, a sequence of interest is identified and isolated from a plasmid library in a single step using, for example, long-range PCR. Following isolation of this sequence, a second polynucleotide that will disrupt the target sequence can be readily inserted between two regions encoding the sequence of interest. In accordance with this aspect, the construct is generated in two steps by (1) amplifying (for example, using long-range PCR) sequences homologous to the target sequence, and (2) inserting another polynucleotide (for example a selectable marker) into the PCR product so that it is flanked by the homologous sequences. Typically, the vector is a plasmid from a plasmid genomic library. The completed construct is also typically a circular plasmid.

In another embodiment, the targeting construct is designed in accordance with the regulated positive selection method described in U.S. patent application Ser. No. 09/954,483, filed Sep. 17, 2001, which is now published U.S. Patent Publication No. 20030032175, the disclosure of which is incorporated herein in its entirety. The targeting construct is designed to include a PGK-neo fusion gene having two lacO sites, positioned in the PGK promoter and an NLS-lacI gene comprising a lac repressor fused to sequences encoding the NLS from the SV40 T antigen. In another embodiment, the targeting construct may contain more than one selectable maker gene, including a negative selectable marker, such as the herpes simplex virus tk (HSV-tk) gene. The negative selectable marker may be operatively linked to a promoter and a polyadenylation signal. (see, e.g., U.S. Pat. Nos. 5,464,764; 5,487,992; 5,627,059; and U.S. Pat. No. 5,631,153).

Once an appropriate targeting construct has been prepared, the targeting construct may be introduced into an appropriate host cell using any method known in the art. Various techniques may be employed in the present disclosure, including, for example, pronuclear microinjection; retrovirus mediated gene transfer into germ lines; gene targeting in embryonic stem cells; electroporation of embryos; sperm-mediated gene transfer; and calcium phosphate/DNA co-precipitates, microinjection of DNA into the nucleus, bacterial protoplast fusion with intact cells, transfection, polycations, e.g., polybrene, polyomithine, etc., or the like (see, e.g., U.S. Pat. No. 4,873,191; Van der Putten, et al., 1985, Proc. Natl. Acad. Sci., USA 82:6148-6152; Thompson, et al., 1989, Cell 56:313-321; Lo, 1983, Mol Cell. Biol. 3:1803-1814; Lavitrano, et al., 1989, Cell, 57:717-723). Various techniques for transforming mammalian cells are known in the art. (see, e.g., Gordon, 1989, Intl. Rev. Cytol., 115:171-229; Keown et al., 1989, Methods in Enzymology; Keown et al., 1990, Methods and Enzymology, Vol. 185, pp. 527-537; Mansour et al., 1988, Nature, 336:348-352).

Selected cells are then injected into a blastocyst (or other stage of development suitable for the purposes of creating a viable animal, such as, for example, a morula) of an animal (e.g., a mouse) to form chimeras (see e.g., Bradley, A. in Teratocarcinomas and Embryonic Stem Cells: A Practical Approach, E. J. Robertson, ed., IRL, Oxford, pp. 113-152 (1987)). Alternatively, selected ES cells can be allowed to aggregate with dissociated mouse embryo cells to form the aggregation chimera. A chimeric embryo can then be implanted into a suitable pseudopregnant female foster animal and the embryo brought to term Chimeric progeny harbouring the homologously recombined DNA in their germ cells can be used to breed animals in which all cells of the animal contain the homologously recombined DNA. In one embodiment, chimeric progeny mice are used to generate a mouse with a heterozygous disruption in the PXR gene. Heterozygous transgenic mice can then be mated. It is well know in the art that typically ¼ of the offspring of such matings will have a homozygous disruption in the PXR gene.

In one embodiment, the phenotype (or phenotypic change) associated with a disruption in the PXR gene is placed into or stored in a database. Preferably, the database includes: (i) genotypic data (e.g., identification of the disrupted gene) and (ii) phenotypic data (e.g., phenotype(s) resulting from the gene disruption) associated with the genotypic data. The database is preferably electronic.

The present disclosure further contemplates conditional transgenic or knockout animals, such as those produced using recombination methods. Bacteriophage P1 Cre recombinase and flp recombinase from yeast plasmids are two non-limiting examples of site-specific DNA recombinase enzymes that cleave DNA at specific target sites (lox P sites for cre recombinase and frt sites for flp recombinase) and catalyze a ligation of this DNA to a second cleaved site. A large number of suitable alternative site-specific recombinases have been described, and their genes can be used in accordance with the method of the present disclosure. Such recombinases include the Int recombinase of bacteriophage .lamda. (with or without Xis) (Weisberg, R. et al., in Lambda II, (Hendrix, R., et al., Eds.), Cold Spring Harbor Press, Cold Spring Harbor, N.Y., pp. 211-50 (1983), herein incorporated by reference); TpnI and the .beta.-lactamase transposons (Mercier, et al., J Bacteriol., 172:3745-57 (1990)); the Tn3 resolvase (Flanagan & Fennewald J. Molec. Biol., 206:295-304 (1989); Stark, et al., Cell, 58:779-90 (1989)); the yeast recombinases (Matsuzaki, et al., J Bacteriol., 172:610-18 (1990)); the B. subtilis SpoIVC recombinase (Sato, et al., J Bacteriol. 172:1092-98 (1990)); the Flp recombinase (Schwartz & Sadowski, J Molec. Biol., 205:647-658 (1989); Parsons, et al., J Biol. Chem., 265:4527-33 (1990); Golic & Lindquist, Cell, 59:499-509 (1989); Amin, et al., J Molec. Biol., 214:55-72 (1990)); the Hin recombinase (Glasgow, et al., J Biol. Chem., 264:10072-82 (1989)); immunoglobulin recombinases (Malynn, et al., Cell, 54:453-460 (1988)); and the Cin recombinase (Haffter & Bickle, EMBO J, 7:3991-3996 (1988); Hubner, et al., J Molec. Biol., 205:493-500 (1989)), all herein incorporated by reference. Such systems are discussed by Echols (J. Biol. Chem. 265:14697-14700 (1990)); de Villartay (Nature, 335:170-74 (1988)); Craig, (Ann. Rev. Genet., 22:77-105 (1988)); Poyart-Salmeron, et al., (EMBO J 8:2425-33 (1989)); Hunger-Bertling, et al., (Mol Cell. Biochem., 92:107-16 (1990)); and Cregg & Madden (Mol. Gen. Genet., 219:320-23 (1989)), all herein incorporated by reference. Cre has been purified to homogeneity, and its reaction with the loxP site has been extensively characterized (Abremski & Hess J Mol. Biol. 259:1509-14 (1984), herein incorporated by reference). Cre protein has a molecular weight of 35,000 and can be obtained commercially from New England Nuclear/Du Pont. The cre gene (which encodes the Cre protein) has been cloned and expressed (Abremski, et al., Cell 32:1301-11 (1983), herein incorporated by reference). The Cre protein mediates recombination between two loxP sequences (Sternberg, et al., Cold Spring Harbor Symp. Quant. Biol. 45:297-309 (1981)), which may be present on the same or different DNA molecule. Because the internal spacer sequence of the loxP site is asymmetrical, two loxp sites can exhibit directionality relative to one another (Hoess & Abremski Proc. Natl. Acad. Sci. U.S.A. 81:1026-29 (1984)). Thus, when two sites on the same DNA molecule are in a directly repeated orientation, Cre will excise the DNA between the sites (Abremski, et al., Cell 32:1301-11 (1983)). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequences flanked by the loxP sites. In addition, recombinase action can result in reciprocal exchange of regions distal to the target site when targets are present on separate DNA molecules.

In one embodiment, purified recombinase enzyme is provided to the cell by direct microinjection. In another embodiment, recombinase is expressed from a co-transfected construct or vector in which the recombinase gene is operably linked to a functional promoter. An additional aspect of this embodiment is the use of tissue-specific or inducible recombinase constructs that allow the choice of when and where recombination occurs.

The cell- and animal-based systems described herein can be utilized as models for diseases Animals of any species, including, but not limited to, mice, rats, rabbits, guinea pigs, pigs, micro-pigs, goats, and non-human primates, e.g., baboons, monkeys, and chimpanzees may be used to generate disease animal models. In addition, cells from humans may be used. These systems may be used in a variety of applications. Such assays may be utilized as part of screening strategies designed to identify agents, such as compounds that are capable of ameliorating disease symptoms. Thus, the animal- and cell-based models may be used to identify drugs, pharmaceuticals, therapies and interventions that may be effective in treating disease. Cell-based systems may be used to identify compounds that may act to ameliorate disease symptoms. For example, such cell systems may be exposed to a compound suspected of exhibiting an ability to ameliorate disease symptoms, at a sufficient concentration and for a time sufficient to elicit such an amelioration of disease symptoms in the exposed cells. After exposure, the cells are examined to determine whether one or more of the disease cellular phenotypes has been altered to resemble a more normal or more wild type, non-disease phenotype.

In addition, animal-based disease systems, such as those described herein, may be used to identify compounds capable of ameliorating disease symptoms. Such animal models may be used as test substrates for the identification of drugs, pharmaceuticals, therapies, and interventions that may be effective in treating a disease or other phenotypic characteristic of the animal. For example, animal models may be exposed to a compound or agent suspected of exhibiting an ability to ameliorate disease symptoms, at a sufficient concentration and for a time sufficient to elicit such an amelioration of disease symptoms in the exposed animals. The response of the animals to the exposure may be monitored by assessing the reversal of disorders associated with the disease. Exposure may involve treating mother animals during gestation of the model animals described herein, thereby exposing embryos or fetuses to the compound or agent that may prevent or ameliorate the disease or phenotype. Neonatal, juvenile, and adult animals can also be exposed.

More particularly, using the animal models of the disclosure, specifically, transgenic mice, methods of identifying agents, including compounds are provided, preferably, on the basis of the ability to affect at least one phenotype associated with a disruption in a PXR gene. In one embodiment, the present disclosure provides a method of identifying agents having an effect on PXR expression or function. The method includes measuring a physiological response of the animal, for example, to the agent, and comparing the physiological response of such animal to a control animal, wherein the physiological response of the animal comprising a disruption in a PXR as compared to the control animal indicates the specificity of the agent. A “physiological response” is any biological or physical parameter of an animal that can be measured. Molecular assays (e.g., gene transcription, protein production and degradation rates), physical parameters (e.g., exercise physiology tests, measurement of various parameters of respiration, measurement of heart rate or blood pressure, measurement of bleeding time, aPTT.T, or TT), and cellular assays (e.g., immunohistochemical assays of cell surface markers, or the ability of cells to aggregate or proliferate) can be used to assess a physiological response. The transgenic animals and cells of the present disclosure may be utilized as models for diseases, disorders, or conditions associated with phenotypes relating to a disruption in a PXR.

The present disclosure provides a unique animal model for testing and developing new treatments relating to BD. Analysis of the BD phenotype allows for the development of an animal model useful for testing, for instance, the efficacy of proposed pharmacological therapies for BD.

Screening Methods

The present disclosure may be employed in a process for screening for agents such as agonists, e.g., agents that bind to and activate PXR polypeptides, or antagonists, e.g., inhibit the activity or interaction of PXR polypeptides with its ligand. Thus, polypeptides of the disclosure may also be used to assess the binding of small molecule substrates and ligands in, for example, cells, cell-free preparations, chemical libraries, and natural product mixtures as known in the art. Any methods routinely used to identify and screen for agents that can modulate receptors may be used in accordance with the present disclosure.

In one embodiment, compositions identified in the screening methods will be classified at least according to the following five characteristics in comparison to rifaximin: solubility, absorption, bacterial penetration, RNA polymerase inhibition, and/or PXR specificity. Each of these characteristics may be different from the properties of rifaximin in one or more of these characteristics and may also be in any combination of the characteristics.
Solubility: Exemplary molecules indentified in the screening assays may have, for example, increased solubility for improved delivery to the colon. If a more absorbed molecule is desired, increased and improved solubility will also be useful for increases systemic absorption.
Absorption: It may be desirable to have increased or improved systemic antibacterial activity. Exemplary molecules indentified in the screening assays may have, for example, improved absorption.
Bacterial Penetration: It may be desirable to have increased or improved bacterial penetration, which may lead to improved antibacterial activity. It may also be useful, if an optimized anti-inflammatory molecule is desired to have decreased bacterial penetration.
RNA Pol. Inhibition: It may be desirable to have increased or improved antibacterial properties, which may be measured as an increase in the RNA pol activity of a compound selected in the screen. It may also be useful, if an optimized anti-inflammatory molecule is desired to have a decreased RNA polymerase inhibition activity.
PXR Specificity: It may be desirable to have increased or improved specificity as measured by binding, activation, and/or downstream signal transduction pathway response. A compound having greater PXR specificity may possibly be dosed at a lower level or may elicit a greater PXR effect or have improved anti-inflammatory properties.

One of skill in the art, having the benefit of this disclosure, would understand assays to test the five characteristics of compounds indentified in the screening assays in comparison to rifaximin. The compounds may also be compared to one another once a few exemplary compounds are identified by the screening assay.

The present disclosure provides methods for identifying and screening for agents that modulate PXR expression or function. More particularly, cells that contain and express PXR gene sequences may be used to screen for therapeutic agents. Such cells may include non-recombinant monocyte cell lines, such as U937 (ATCC# CRL-1 593), THP-1 (ATCC# TIB-202), and P388D1 (ATCC# TIB-63); DPX2; endothelial cells such as HUVEC's and bovine aortic endothelial cells (BAEC's); as well as generic mammalian cell lines such as HeLa cells and COS cells, e.g., COS-7 (ATCC# CRL-1651). Further, such cells may include recombinant, transgenic cell lines. For example, the transgenic mice of the disclosure may be used to generate cell lines, containing one or more cell types involved in a disease that can be used as cell culture models for that disorder. While cells, tissues, and primary cultures derived from the disease transgenic animals of the disclosure may be utilized, the generation of continuous cell lines is certain. For examples of techniques that may be used to derive a continuous cell line from the transgenic animals, see Small, et al., Mol. Cell Biol., 5:642-48 (1985).

PXR gene sequences may be introduced into, and overexpressed in, the genome of the cell of interest. In order to overexpress a PXR gene sequence, the coding portion of the PXR gene sequence may be ligated to a regulatory sequence that is capable of driving gene expression in the cell type of interest. Such regulatory regions will be well known to those of skill in the art, and may be utilized in the absence of undue experimentation. PXR gene sequences may also be disrupted or underexpressed. Cells having PXR gene disruptions or underexpressed PXR gene sequences may be used, for example, to screen for agents capable of affecting alternative pathways that compensate for any loss of function attributable to the disruption or underexpression.

In vitro systems may be designed to identify compounds capable of binding the PXR gene products. Such compounds may include, but are not limited to, peptides made of D- and/or L-configuration amino acids (in, for example, the form of random peptide libraries; (see e.g., Lam, et al., Nature, 354:82-4 (1991)), phosphopeptides (in, for example, the form of random or partially degenerate, directed phosphopeptide libraries; see, e.g., Songyang, et al., Cell, 72:767-78 (1993)), antibodies, and small organic or inorganic molecules. Compounds identified may be useful, for example, in modulating the activity of PXR gene proteins, preferably mutant PXR gene proteins; elaborating the biological function of the PXR gene protein; or screening for compounds that disrupt normal PXR gene interactions or themselves disrupt such interactions.

For example, an assay used to identify compounds that bind to the PXR gene protein involves preparing a reaction mixture of the PXR gene protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring the PXR gene protein or the test substance onto a solid phase and detecting target protein/test substance complexes anchored on the solid phase at the end of the reaction. In one embodiment of such a method, the PXR gene protein may be anchored onto a solid surface, and the test compound, which is not anchored, may be labeled, either directly or indirectly.

Microtitre plates may be conveniently utilized. The anchored component may be immobilized by non-covalent or covalent attachments. Non-covalent attachment may be accomplished simply by coating the solid surface with a solution of the protein and drying. Alternatively, an immobilized antibody, preferably a monoclonal antibody, specific for the protein may be used to anchor the protein to the solid surface. The surfaces may be prepared in advance and stored.

To conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously nonimmobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the previously nonimmobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for PXR gene product or the test compound to anchor any complexes formed in solution, and a labeled antibody specific for the other component of the possible complex to detect anchored complexes.

Compounds that are shown to bind to a particular PXR gene product through one of the methods described above can be further tested for their ability to elicit a biochemical response from the PXR gene protein. Agonists, antagonists and/or inhibitors of the expression product can be identified utilizing assays well known in the art.

A variety of methods may be employed to diagnose disease conditions associated with the PXR gene. Specifically, reagents may be used, for example, for the detection of the presence of PXR gene mutations, or the detection of either over or under expression of PXR gene mRNA.

The following examples are intended only to illustrate the present disclosure and should in no way be construed as limiting the subject disclosure.

### EXAMPLES

### Materials and Methods

### Chemicals

Rifampicin (RIF), 3-(4-Methylpiperazinyliminomethyl)rifamycin SV; Rifaximin (RIFax), 4-Deoxy-4′-methylpyrido[1′,2′-1,2]imidazo[5,4-c]rifamycin SV; and midazolam (MDZ) were obtained from Sigma-Aldrich (St. Louis, Mo.). 1′-Hydroxymidazolam (1′-OH-MDZ) was purchased from BD Gentest (Woburn, Mass.). All other chemicals were of the highest grade commercially available.

Animals and Treatments

PXR-humanized (hPXR), Pxr-null and wild-type (WT) mice were maintained under a standard 12 h light/12 h dark cycle with water and chow provided ad libitum. Pxr-null and hPXR mice were described previously (Staudinger et al., 2001; Ma et al., 2007). To investigate the potential role of RIFax in PXR activation, 2-4 month old male hPXR, Pxr-null and WT mice were treated orally with 25 mg/kg/day of RIFax for 3 days. RIF, a specific human PXR ligand, was used as positive control at 25 mg/kg/day (p.o.) for 3 days. Corn oil was used as vehicle for both RIF and RIFax treatment. All mice were killed by CO₂ asphyxiation 24 h after the last dose. Liver and small intestine were collected and frozen at −80° C. for further analysis.

RIFax Pharmacokinetics and its Distribution in Intestinal Tract

For pharmacokinetic analysis, WT, Pxr-null, and hPXR mice were treated with 10 mg/kg RIF or RIFax by oral gavage. Corn oil was used as vehicle for both RIF and RIFax treatment. Blood samples were collected from suborbital veins using heparinized tubes at pre-dose, 0.25, 0.5, 1.5, 3, 6, 9, 12, 24 and 48 h after the administration. To compare the metabolic profiles of RIFax and RIF, 10 mg/kg RIFax and RIF were administered by intravenous (i.v.) and intraperitoneal (i.p.). For i.p. injections, corn oil was used as vehicle for both RIF and RIFax, and blood samples were collected from suborbital veins at pre-dose, 0.25, 0.5, 1, 2, 4, 8, 24 and 48 h after the administration. For i.v. injections, 30% polyethylene glycol (PEG, Wt. 400) was used as vehicle for both RIF and RIFax, and blood samples were collected from suborbital veins at pre-dose, 0.0833, 0.25, 0.5, 1, 2, 4, 8, 24 and 48 h after the administration. Serum was separated by centrifugation at 8,000 g for 10 min Fifty μl of serum was mixed with 150 μl methanol, vortexed twice for 20 s, and centrifuged at 14,000 rpm for 10 min at 4° C. The upper organic layer was then transferred to an auto-sampler vial for RIF or RIFax detection by LC-MS/MS using an API2000 SCIEX triple-quadrupole tandem mass spectrometer (Applied Biosystems/MDS Sciex, Foster City, Calif.). Pharmacokinetic parameters of RIF and RIFax were estimated from the serum concentration-time data by a non-compartmental approach using WinNonlin (Pharsight, Mountain View, Calif.). The maximum concentration in serum (Cₘₐₓ) was obtained from the original data. The area under the serum concentration-time curve (AUC_{0-48 h}) was calculated by the trapezoidal rule. To detect the distribution in intestinal tract, mice were treated with 10 mg/kg RIFax or RIF (p.o.). At 1.5, 3, 6, 9, 12, 24 and 48 h after the administration, the mice were killed and the contents in different segments of the intestinal tract were collected. Intestinal contents were weighted, and homogenized in methanol (100 mg/ml). The homogenate was centrifuged at 14,000 rpm for 10 min at 4° C. The upper organic layer was then transferred to an auto-sampler vial for RIF or RIFax detection by LC-MS/MS.

Analysis of RIFax and RIF by LC-MS/MS

RIFax and RIF were determined by LC-MS/MS, carried out using a high-performance liquid chromatography system consisting of a PerkinElmer Series 200 quaternary pump, vacuum degasser, and autosampler with a 100 μl loop interfaced to LC-MS/MS as noted above. RIFax and RIF were separated on a Luna C18 50 mm×4.6 mm i d column (Phenomenex, Torrance, Calif.). The flow rate through the column at ambient temperature was 0.25 ml/min with 85% methanol and 15% H₂O containing 0.1% formic acid. The mass spectrometer was operated in the turbo ion spray mode with positive ion detection. The turbo ion spray temperature was maintained at 300° C., and a voltage of 4.8 kV was applied to the sprayer needle. N₂ was used as the turbo ion spray and nebulizing gas. The detection and quantification were performed using the multiple reactions monitoring (MRM) mode, with m/z 786.3/754.5 for RIFax, and m/z 823.5/791.5 for RIF.

Pharmacokinetic Analysis of MDZ in hPXR Mice Pretreated with RIFax

hPXR mice were pretreated with or without 10 mg/kg RIFax, once daily for 3 days. Corn oil was used as the vehicle for RIFax treatment. Twenty-four hours after the last dose of RIFax, mice were administered 2.5 mg/kg MDZ by oral gavage. Blood samples were collected from suborbital veins using heparinized tubes at pre-dose, 5, 10, 20, 30, 60, and 90 min after administration of MDZ. Serum was separated by centrifugation at 8,000 g for 10 min. For MDZ and 1′-OH-MDZ extraction, 50 μl of serum was mixed with 150 μl of phosphate-buffered saline, 200 μl of ethyl acetate, and 200 μl of methyl t-butyl ether. The mixture was centrifuged at 3,000 rpm for 5 min at 4° C. The organic layer was then transferred to a new tube, dried with N₂, and reconstituted in 100 μl of 70% aqueous methanol and 30% H₂O containing 0.1% formic acid. MDZ and 1′-OH-MDZ were detected by LC-MS/MS, as described previously (Ma et al., 2007). Pharmacokinetic parameters for MDZ and 1′-OH-MDZ were estimated from the plasma concentration-time data by a non-compartmental approach using WinNonlin (Pharsight, Mountain View, Calif.). The area under the serum concentration-time curve (AUC_{0-90 min}) was calculated by the trapezoidal rule. The maximum concentration in serum (Cₘₐₓ) and its corresponding time (Tₘₐₓ) were obtained from the original data.

qPCR Analysis of PXR Target Genes

The following PXR target genes were analyzed by quantitative real-time PCR (qPCR): cytochrome P450 3A11 (CYP3A11), glutathione S-transferase alpha 1 (GSTA1), multidrug resistance protein 2 (MRP2), and organic anion transporting polypeptide 2 (OATP2) (Guo et al., 2002; Kast et al., 2002; Rosenfeld et al., 2003). RNA was extracted from different tissues using TRIzol reagent (Invitrogen, Carlsbad, Calif.). qPCR was performed using cDNA generated from 1 μg total RNA with SuperScript II Reverse Transcriptase kit (Invitrogen). Primers were designed for qPCR using the Primer Express software (Applied Biosystems); primer sequences are listed in Table 1. PCR reactions were carried out using SYBR green PCR master mix (SuperArray) in an ABI Prism 7900HT Sequence Detection System (Applied Biosystems, Foster City, Calif.). Values were quantified using the Comparative CT method, and samples normalized to β-actin.

TABLE 1
Primer sequences for qPCR analysis.

| | Primer sequences |
|---|---|
| CYP3A11 | Fwd: 5′-AGC AGG GAT GGA CCT GG-3′ |
| | (SEQ ID NO: 1) |
| | Rev: 5′-CGG TAG AGG AGC ACC AA-3′ |
| | (SEQ ID NO: 2) |
| GSTA1 | Fwd: 5′-CAG CCT GGC AGC CAG AGA-3′ |
| | (SEQ ID NO: 3) |
| | Rev: 5′-TCT GTG GCT CCA TCA ATG CA-3′ |
| | (SEQ ID NO: 4) |
| MRP2 | Fwd: 5′-CGT GGC TGT TGA GCG AAT AA-3′ |
| | (SEQ ID NO: 5) |
| | Rev: 5′-TCT CAC CTT TTT TGG GCC AAT-3′ |
| | (SEQ ID NO: 6) |
| OATP2 | Fwd: 5′-TGC TGA CTG CAA CAC AAA GTG T-3′ |
| | (SEQ ID NO: 7) |
| | Rev: 5′-AGC TGA CAT GTA TGA TAG ACC ATT |
| | GTC-3′ |
| | (SEQ ID NO: 8) |

Cell-Based Reporter Assay

A hepG2 cell line (DPX2) with stable expression of recombinant human PXR and a PXR-response element cloned in a luciferase vector was obtained from Puracyp Inc. (Carlsbad, Calif.). The construction and validation of the cell lines were reported previously (Yueh et al., 2005). The cells were seeded according to the distributor's instruction. RIFax (1, 10, 100 μM) was added to the culturing medium and 10 μM RIF used as positive control. The activation of PXR was determined by measuring the firefly luciferase activity 24 h later, followed by normalization of luciferase activity by protein concentrations. For cell-based reporter assay of nuclear receptors CAR, PPARα, PPARγ, and FXR, HCT116 cells were plated on 24-well plates (5×104 cells/well, cultured in DMEM containing 10% FBS), and transfected with the various expression vectors using Fugene transfection reagent (Roche, Indianapolis, Ind.). The mouse PPAR and CAR vectors were described in previous reports (Kliewer et al., 1992; Swales et al., 2005). The mouse FXR vector was provided by Dr Christopher J. Sinal. After 24 h post-transfection, the cells were incubated with vehicle (DMSO) and 10 μM RIFax for 24 h. TCPOBOP (250 nM), Wy-14,643 (10 μM), rosiglitazone (10 μM), and GW4064 (25 μM) were used as positive controls respectively for mouse CAR, PPARα, PPARγ, and FXR. A standard dual luciferase assay was used and normalized to a cotransfected control reporter (Promega, Madison, Wis.). Each in vitro assay was repeated at least three times.

Statistical Analysis

All values are expressed as the mean±SD and analyzed by two-tailed Student's t test. p<0.05 was regarded as significantly different between groups.

The PXR-humanized (hPXR), Pxr-null, and wild-type mice were treated orally with rifaximin, and rifampicin, a well-characterized human PXR ligand. Rifaximin was highly concentrated in the intestinal tract when compared to rifampicin. Rifaximin treatment resulted in significant induction of PXR target genes in the intestine of hPXR mice, but not in wild-type and Pxr-null mice. However, rifaximin treatment demonstrated no significant effect on hepatic PXR target genes in wild-type, Pxr-null, and hPXR mice. Consistent with the in vivo data, cell-based reporter gene assay revealed rifaximin-mediated activation of human PXR, but not the other xenobiotic nuclear receptors CAR, PPARα, PPARγ, and FXR. Pretreatment with rifaximin did not affect the pharmacokinetics of the CYP3A substrate midazolam, but increased the Cₘₐₓ and decreased Tₘₐₓ of 1′-hydroxymidazolam. Collectively, the current study identified rifaximin as a gut-specific human PXR ligand

Metabolic Profiles and Intestinal Tract Distribution of RIFax in Mice

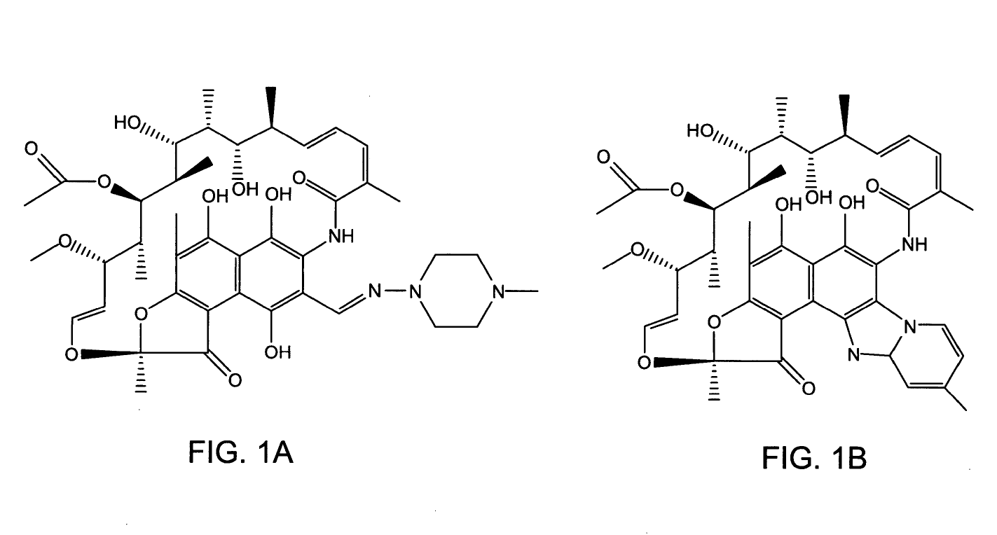

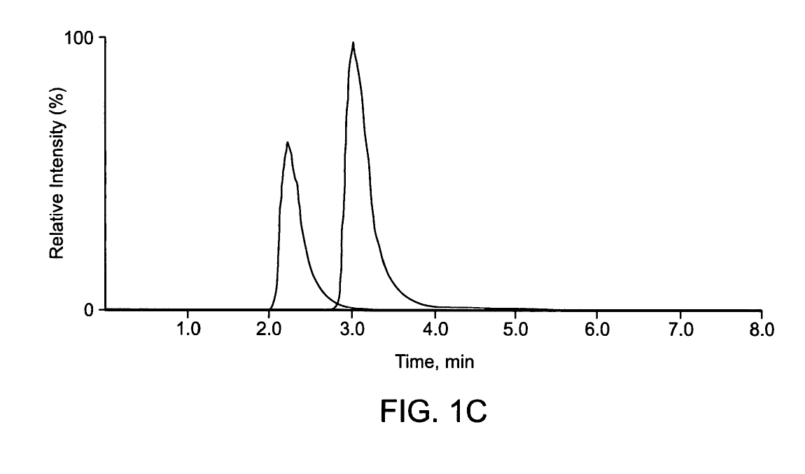

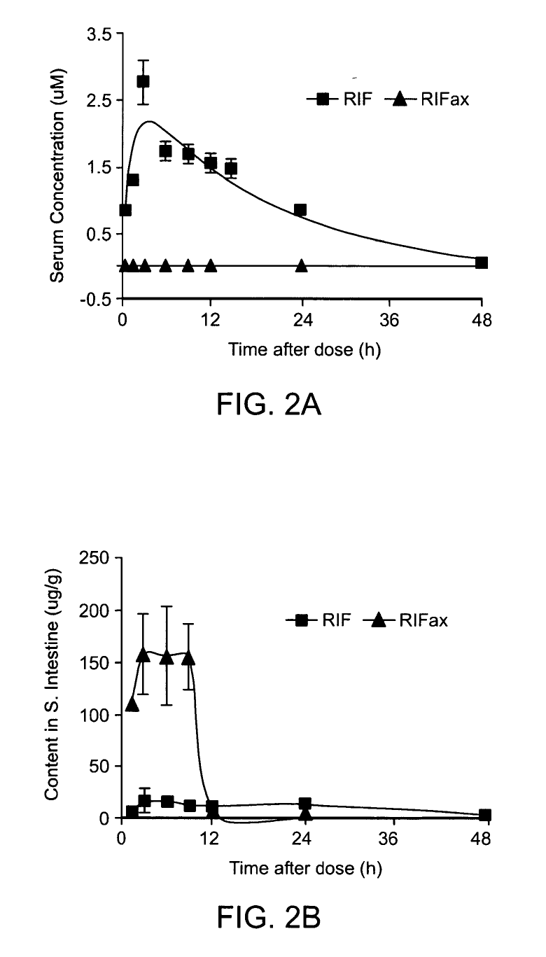

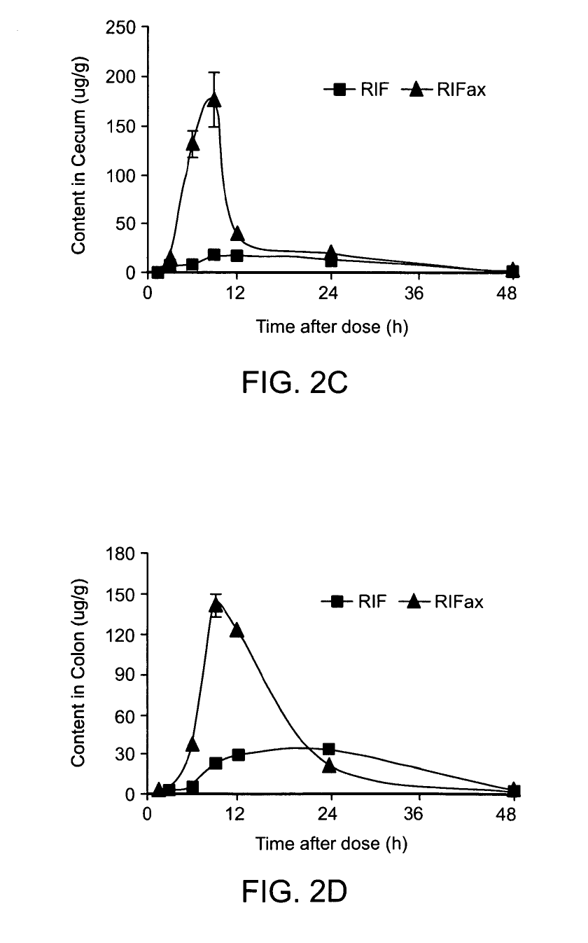

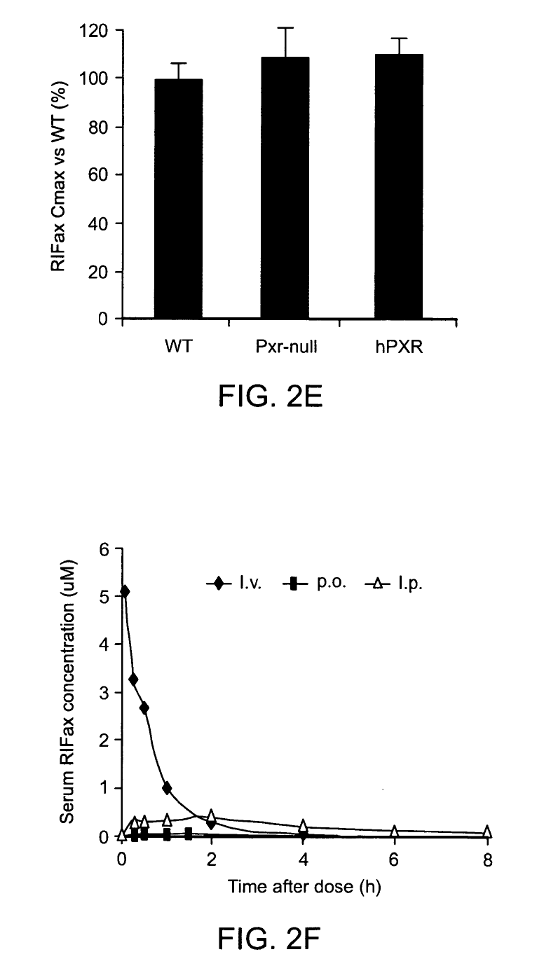

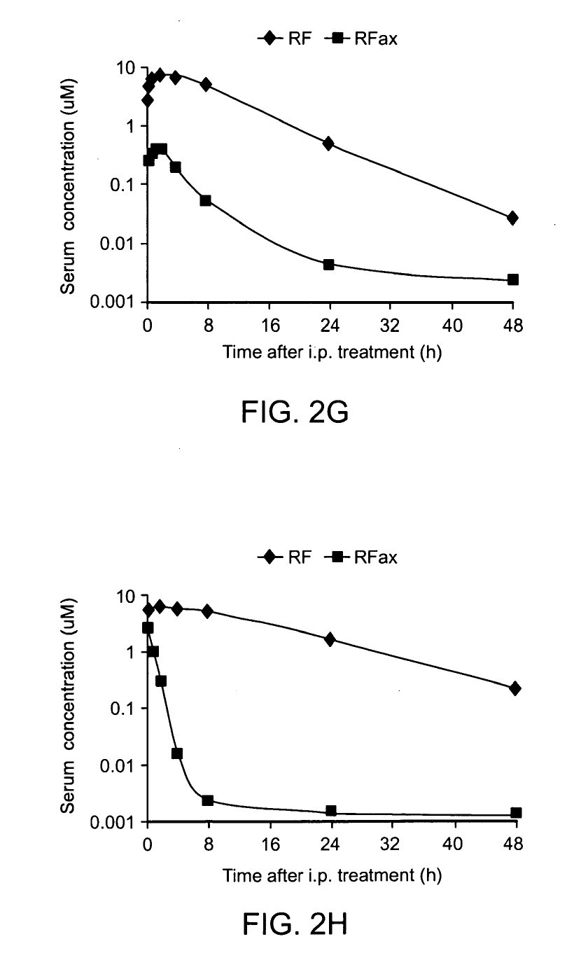

LC-MS/MS was used to study the pharmacokinetics of RIF and RIFax. The retention time was 2.21 min for RIF, m/z 823.5/791.5 (Peak 1 in FIG. 1C), and 3.03 min for RIFax, m/z 786.3/754.5 (Peak 2 in FIG. 1C). The detection limit was 0.023 pmol for RIF, and 0.012 pmol for RIFax. After a single oral dose of RIF or RIFax, mouse blood samples and intestinal contents were collected at different time points up to 48 h following treatment. In the pharmacokinetic study, the Cₘₐₓ of serum RIFax was 0.04 μM, ˜70-fold lower than that of RIF (2.75 μM). The AUC_{0-48 h} of serum RIFax was ˜300-fold lower than that of RIF (FIG. 2A). However, for intestinal tract distribution, the RIFax concentration was significantly higher than that of RIF. In the small intestine, RIF concentration was below 20 μg/g at all time-points measured (FIG. 2B). For RIFax, the concentration was ˜160 μg/g and lasted 9 h following administration. The RIFax intestinal tract distribution in the cecum (FIG. 2C) and colon (FIG. 2D) was similar to that of the small intestine. No significant difference in RIFax metabolism was found among WT, Pxr-null and hPXR mice after oral treatment. The Cₘₐₓ of RIFax (p.o. treatment) in WT, Pxr-null and hPXR mice are shown in FIG. 2E. RIFax is well known as non-absorbable rifamycin by oral treatment. By i.p. injection, RIFax was not well absorbed, and the bioavailability was significant lower than that of RIF (FIGS. 2F, 2G). Differences in metabolic profiles between RIFax and RIF were observed after i.v. treatment, as ultra-short T_{1/2} and -low AUC for RIFax when compared with RIF (FIG. 2H).

PXR Activation by RIFax

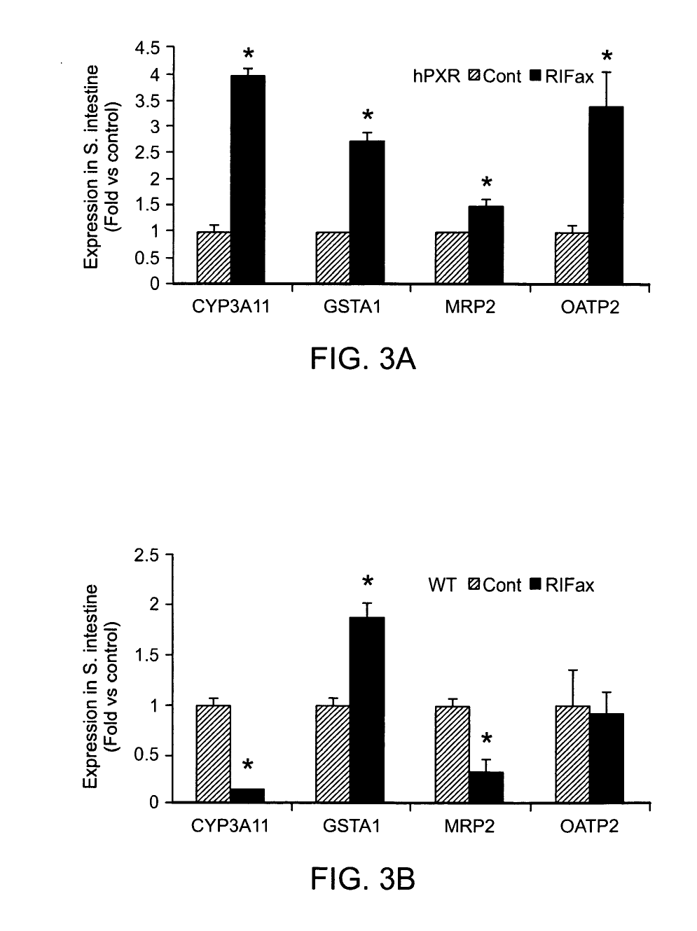

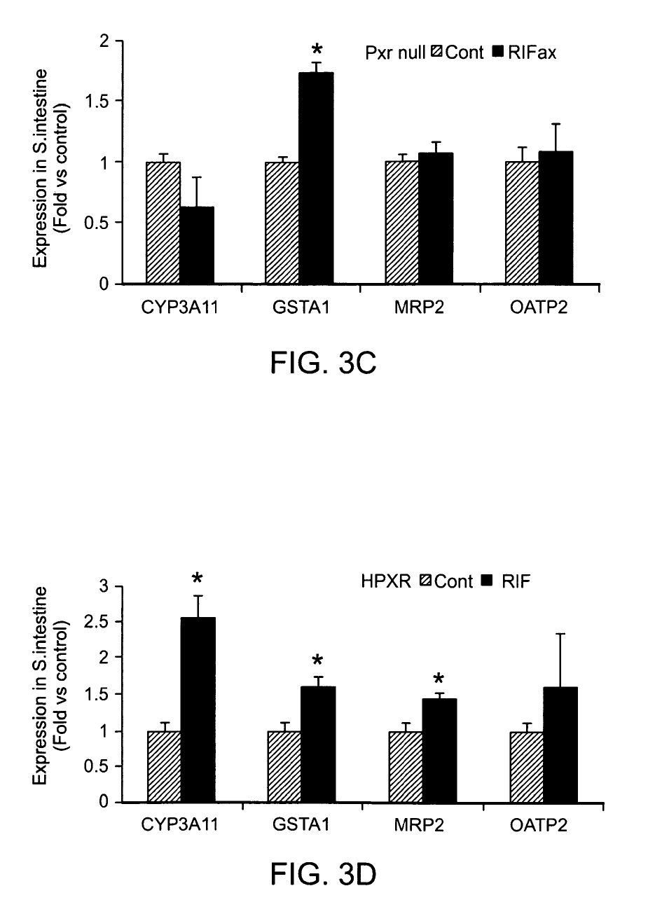

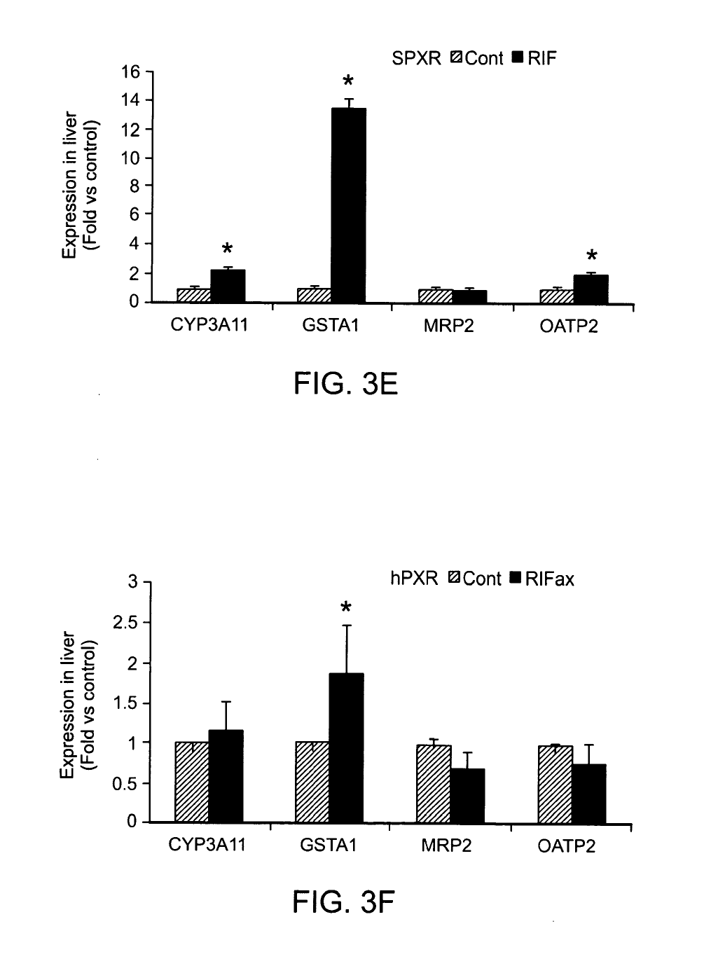

PXR was detected in duodenum, jejunum, ileum, cecum, and colon, but not in stomach of WT and hPXR mice (Ma et al., 2007). Due to the high distribution of RIFax in the intestinal tract and expression of PXR in the gut, the effect of RIFax on gut PXR target genes was investigated by qPCR. In the small intestine of hPXR mice treated with RIFax, CYP3A11, GSTA1, MRP2 and OATP2 were all up-regulated (FIG. 3A). Intestinal CYP3A11 was increased ˜4-fold compared to vehicle-treated hPXR mice, while expression was inhibited in WT mice and no significant change observed in Pxr-null mice (FIGS. 3B and 3C). Intestinal GSTA1 mRNA was up-regulated in all the three mouse strains after RIFax treatment, with 87%, 74%, and 172% increases noted in WT, Pxr-null, and hPXR mice, suggesting that Gsta1 gene may not be a direct PXR target but may be elevated by an indirect mechanism. Without wishing to be bound by any particular scientific theory, one possible explanation for the effect of RIFax on GSTA1 is the antibiotic activity of RIFax. RIFax was treated orally at 25 mg/kg for 3 days, which may change the gut bacterial homeostasis, and indirectly effect GSTA1 expression. A significant up-regulation of intestinal MRP2 mRNA was noted in hPXR mice following RIFax treatment, whereas its expression was significantly suppressed in WT mice, with no change observed in Pxr-null mice (FIGS. 3B and 3C). MRP2, which was reported to be activated by RIF and PCN in human and rat hepatocytes, respectively (Kast et al., 2002), was not markedly induced by RIF in liver and only modestly induced by RIF or RIFax in the gut. Others found that MRP2 is not significantly induced by mouse PXR ligands such as PCN (Maher et al., 2005). The finding that MRP2 is not induced by RIF in the hPXR mice suggests a possible species difference in the cis elements controlling the Mrp2 gene between humans and mice. Intestinal OATP2 mRNA was increased 3.4-fold in hPXR mice after RIFax treatment, but no significant induction of this mRNA was noted in both WT and Pxr-null mice (FIGS. 3B and 3C). As expected, RIF also induced the four mRNAs in intestine but the extent of induction was less than that observed with RIFax (FIG. 3D). In contrast, RIF produced a significant induction of CYP3A11, GSTA1, and OATP2 in liver, whereas only GSTA1 mRNA was increased in the liver of RIFax-treated hPXR mice (FIGS. 3E and 3F). These data indicate that RIFax is a gut-specific human PXR ligand.

Human PXR Activation by RIFax in a Cell-Based Reporter Assay

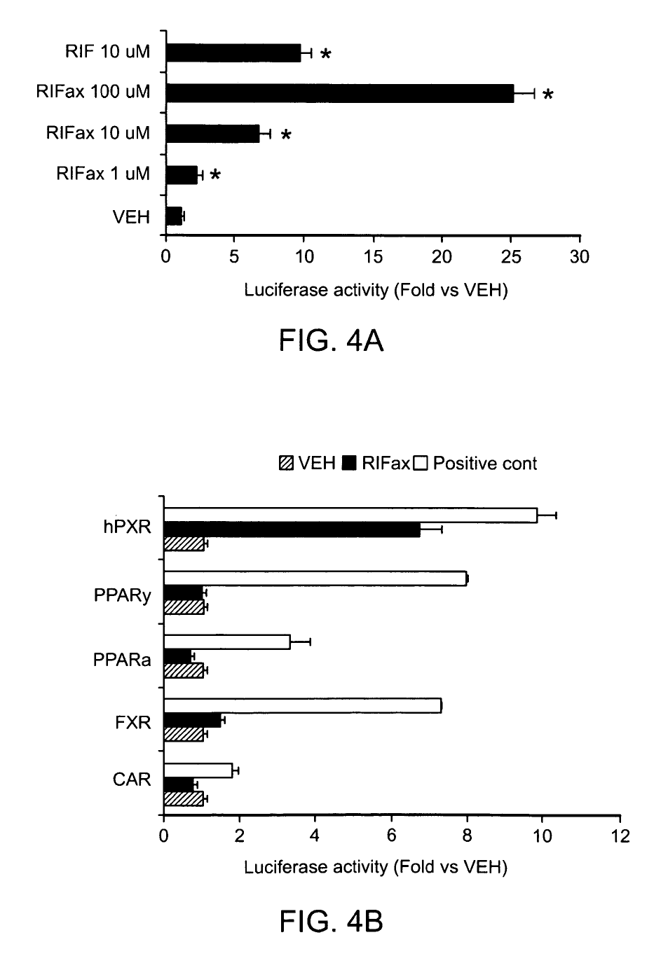

A dose-dependent increase in luciferase activity was observed in a cell-based reporter assay for hPXR activation by RIFax. Incubation with 1, 10, and 100 μM RIFax in the hPXR reporter system produced a 2.1-, 6.7-, and 25.2-fold increase respectively, vs DMSO control (FIG. 4A). RIFax at 100 nM had no significant effect on hPXR while 10 μM RIFax produced no significant change in luciferase activity in the presence of PPARα, PPARγ, CAR and FXR (FIG. 4B).

Pharmacokinetic of MDZ in hPXR Mice Pretreated with RIFax

Following a single oral administration of MDZ (2.5 mg/kg), the serum concentration-time course of MDZ and 1′-OH-MDZ in hPXR mice determined. Pharmacokinetic parameters were estimated by non-compartmental analysis. There were no significant changes (p>0.05) for the Cₘₐₓ, Tₘₐₓ, and AUC_{0-90 min} of MDZ in hPXR mice pretreated with or without RIFax. The RIFax pretreatment in hPXR mice had no significant effect on AUC_{0-90 min} of 1′-OH-MDZ, the major metabolite of MDZ. However, the Cₘₐₓ value of 1′-OH-MDZ was 50% higher (p<0.05) in RIFax pretreated hPXR mice, and the corresponding Tₘₐₓ was significantly shorter than the control group (Table 2). These results suggested that the RIFax-mediated CYP3A11 up-regulation in hPXR mice intestine contributed to extrahepatic first-pass metabolism of MDZ.

Table 2. Pharmacokinetics of MDZ in hPXR mice pretreated with or without RIFax, at 10 mg/kg/day for 3 days. Serum MDZ and 1′-OH-MDZ were detected by LC-MS/MS. AUC_{0-90 min} for MDZ and 1′-OH-MDZ were estimated from the plasma concentration-time data by a non-compartmental approach using WinNonlin (Pharsight, Mountain View, Calif.). Cₘₐₓ and Tₘₐₓ were obtained from the original data. Data are expressed as means±SD, n=3. *p<0.05 compared with control.

TABLE 2

| | Cont | RIFax | RIFax/Cont |
|---|---|---|---|
| MDZ | | | |
| Cₘₐₓ (nmol/L) | 477 ± 40.3 | 383 ± 19.1 | 0.8 |
| Tₘₐₓ (min) | 10.0 ± 0.0 | 12.5 ± 10.6 | 1.3 |
| AUC_{0-90 min} (μmol · min/L) | 8.0 ± 0.4 | 8.9 ± 0.7 | 1.1 |
| 1′-OH-MDZ | | | |
| Cₘₐₓ (nmol/L) | 562 ± 4.9 | 823 ± 55.2* | 1.5 |
| Tₘₐₓ (min) | 25.0 ± 7.1 | 7.5 ± 3.5* | 0.3 |
| AUC_{0-90 min} (μmol · min/L) | 32.4 ± 3.0 | 36.5 ± 4.2 | 1.1 |

The effect of RIFax on PXR was investigated. By using hPXR, Pxr-null, and WT mice, and a cell-based human PXR reporter gene assay, RIFax was identified as a gut-specific human PXR ligand. During the pharmaceutical development of RIFax, CYP3A4 induction by RIFax was noted in a human hepatocyte model. Reported herein, is a novel finding that RIFax is a gut specific human PXR ligand that up-regulates PXR target genes including CYP3A. In the DPX2 cell line with stable recombinant human PXR expression, hPXR was significantly activated at RIFax concentrations over 1 μM, as the luciferase activity increased 2.1 fold vs vehicle. The EC50 for activation of hPXR by RIFax in the DPX2 cell line was estimated around 20 μM. The RIFax concentration in intestine is much higher than 20 μM after RIFax treatments. In the current study, when mice were treated with 10 mg/kg RIFax (single dose, p.o.), the RIFax concentration in the intestinal tract was up to 150 ug/g (about 200 μM) intestinal content. In humans, after 3 days of RIFax treatment (800 mg daily, p.o.), the RIFax concentration was about 8 mg/g (about 10,000 μM) stool (Jiang et al., 2000), which indicated an extremely high concentration of RIFax exposure in the intestine. The effect of RIFax on gut PXR, but not the liver receptor, was probably related to its poor absorption. The metabolic profiles of RIFax in this study are consistent with previous studies, as high concentrations of RIFax in intestinal tract with only minor distribution in the blood (Venturini, 1983; Cellai et al., 1984); this was independent of PXR expression in the gut indicating that lack of absorption is not due to PXR-induced metabolism. In humans, RIFax absorption is also negligible after oral administration. After a single oral dose of RIFax (400 mg), the plasma RIFax concentration was below the detection limit (2 ng/ml). In urine, very small amounts of the unchanged molecule were detected that was <0.01% of the administered dose (Descombe et al., 1994). Thus, the current study indicates that during clinical use, RIFax functions as an antibiotic and also as a PXR activator in the gut.

The identification of RIFax as human PXR ligand provides new insights into the role of RIFax in pharmacology and therapeutics. PXR, a member of the nuclear receptor family of ligand-activated transcription factors, is an integral component of the body's defense mechanism involved in the detoxication of xenobiotics (Kliewer et al., 2002). PXR activation regulates the expression of xenobiotics oxidation and conjugation enzymes, and transporters, involved in the metabolism and elimination of potentially harmful chemicals from the body. Previous studies revealed CYP3A4 induction by RIFax in a human hepatocyte model. Two clinical studies that used MDZ and an oral contraceptive containing ethinyl estradiol and norgestimate (Trapnell et al., 2007) demonstrated that RIFax did not alter the pharmacokinetics of these drugs thus indicating that RIFax had no significant effect on intestinal or hepatic CYP3A4. However, herein, intestinal CYP3A11 was significantly up-regulated in hPXR mice treated with RIFax. In the pharmacokinetics study of MDZ in hPXR pretreated with RIFax, a 20% decrease of Cₘₐₓ was observed, which was consistent with the Cₘₐₓ increase of its major metabolite 1′-OHMDZ, and without wishing to be bound by any particular scientific theory, can be explained by the first-pass effect through intestinal CYP3A metabolism. However, there was no parallel decrease of MDZ AUC in hPXR pretreated with RIFax. AUC is not only related to first-pass elimination, but also other factors, such as absorption. In hPXR mice pretreated with RIFax, several intestinal genes including transporters were up-regulated, such as the influx transporter OATP2, which may contribute to the increase of MDZ absorption. The bioavailability study on MDZ was not performed, because of its poor bioavailability and large variation in mice (Granvil et al., 2003). The beneficial aspects of PXR activation is its role in detoxication by up-regulating the enzymes and transporters involved in elimination of the xenobiotics, including P450s, GST, OATP, MRP, etc. (Kliewer, 2003; Saini et al., 2005; Wagner et al., 2005). PXR target genes are components in intestinal barrier function against xenobiotics and bacteria (Langmann et al., 2004). In the small intestine of hPXR mice treated with RIFax, several PXR target genes such as CYP3A11, GSTA1, MRP2 and OATP2 were up-regulated. RIFax is beneficial in the treatment of multiple chronic gastrointestinal disorders, such as hepatic encephalopathy, intestinal gas and gas-related symptoms, diverticular disease, pouchitis, and BD (Scarpignato and Pelosini, 2005). The mechanisms contributing to the beneficial effects of RIFax in chronic gastrointestinal disorders are not fully understood. subject In a dextran sulfate sodium (DSS)-induced BD mouse model, PCN-mediated PXR activation significantly prevented DSS-induced colitis (Shah et al., 2007), which indicates the potential value of PXR ligands as a therapeutic for BD. Further human studies are suggested to assess the role of RIFax-mediated gut PXR activation in therapeutics of chronic gastrointestinal disorders.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Abbreviations: RIF, rifampicin; RIFax, rifaximin; PXR, pregnane X receptor; CAR, Constitutive Androstane Receptor; PPARα, Peroxisome Proliferator-Activated Receptor alpha; PPARγ, Peroxisome proliferator-activated receptor gamma; FXR, farnesoid X receptor; WT, wild-type mice; hPXR, PXR-humanized mice; CYP3A, cytochrome P450 3A; GSTA, glutathione S-transferase alpha; MRP, multidrug resistance protein; OATP, organic anion transporting polypeptide; MDZ, midazolam.

### REFERENCES

Abdelrazeq A S, Kelly S M, Lund J N and Leveson S H (2005) Rifaximin-ciprofloxacin combination therapy is effective in chronic active refractory pouchitis. Colorectal Dis 7:182-186.
Bertilsson G, Heidrich J, Svensson K, Asman M, Jendeberg L, Sydow-Backman M, Ohlsson R, Postlind H, Blomquist P and Berkenstam A (1998) Identification of a human nuclear receptor defines a new signaling pathway for CYP3A induction. Proc Natl Acad Sci USA 95:12208-12213.
Carnahan V E and Redinbo M R (2005) Structure and function of the human nuclear xenobiotic receptor PXR. Curr Drug Metab 6:357-367.
Cellai L, Colosimo M, Marchi E, Venturini A P and Zanolo G (1984) Rifaximin (L/105), a new topical intestinal antibiotic: pharmacokinetic study after single oral administration of 3H-rifaximin to rats. Chemioterapia 3:373-377.
Descombe J J, Dubourg D, Picard M and Palazzini E (1994) Pharmacokinetic study of rifaximin after oral administration in healthy volunteers. Int J Clin Pharmacol Res 14:51-56.
Di Stefano M, Strocchi A, Malservisi S, Veneto G, Ferrieri A and Corazza G R (2000) Non-absorbable antibiotics for managing intestinal gas production and gas-related symptoms. Aliment Pharmacol Ther 14:1001-1008.
Doherty M M and Charman W N (2002) The mucosa of the small intestine: how clinically relevant as an organ of drug metabolism? Clin Pharmacokinet 41:235-253.
Dring M M, Goulding C A, Trimble V I, Keegan D, Ryan A W, Brophy K M, Smyth C M, Keeling P W, O'Donoghue D, O'Sullivan M, O'Morain C, Mahmud N, Wikstrom A C, Kelleher D and McManus R (2006) The pregnane X receptor locus is associated with susceptibility to inflammatory bowel disease. Gastroenterology 130:341-348; quiz 592.
Gionchetti P, Rizzello F, Lammers K M, Morselli C, Tambasco R and Campieri M (2006) Antimicrobials in the management of inflammatory bowel disease. Digestion 73 Suppl 1:77-85.
Gionchetti P, Rizzello F, Venturi A, Ugolini F, Rossi M, Brigidi P, Johansson R, Ferrieri A, Poggioli G and Campieri M (1999) Antibiotic combination therapy in patients with chronic, treatment-resistant pouchitis. Aliment Pharmacol Ther 13:713-718.
Granvil C P, Yu A M, Elizondo G, Akiyama T E, Cheung C, Feigenbaum L, Krausz K W and Gonzalez F J (2003) Expression of the human CYP3A4 gene in the small intestine of transgenic mice: in vitro metabolism and pharmacokinetics of midazolam. Drug Metab Dispos 31:548-558.
Guo G L, Staudinger J, Ogura K and Klaassen C D (2002) Induction of rat organic anion transporting polypeptide 2 by pregnenolone-16alpha-carbonitrile is via interaction with pregnane X receptor. Mol Pharmacol 61:832-839.
Guslandi M, Petrone M C and Testoni P A (2006) Rifaximin for active ulcerative colitis Inflamm Bowel Dis 12:335.
Jiang Z D, Ke S, Palazzini E, Riopel L and Dupont H (2000) In vitro activity and fecal concentration of rifaximin after oral administration. Antimicrob Agents Chemother 44:2205-2206.
Kast H R, Goodwin B, Tarr P T, Jones S A, Anisfeld A M, Stoltz C M, Tontonoz P, Kliewer S, Willson T M and Edwards P A (2002) Regulation of multidrug resistance-associated protein 2 (ABCC2) by the nuclear receptors pregnane X receptor, farnesoid X-activated receptor, and constitutive androstane receptor. J Biol Chem 277:2908-2915.
Kliewer S A (2003) The nuclear pregnane X receptor regulates xenobiotic detoxification. J Nutr 133:2444 S-2447S.
Kliewer S A, Goodwin B and Willson T M (2002) The nuclear pregnane X receptor: a key regulator of xenobiotic metabolism. Endocr Rev 23:687-702.
Kliewer S A, Umesono K, Noonan D J, Heyman R A and Evans R M (1992) Convergence of 9-cis retinoic acid and peroxisome proliferator signalling pathways through heterodimer formation of their receptors. Nature 358:771-774.
Langmann T, Moehle C, Mauerer R, Scharl M, Liebisch G, Zahn A, Stremmel W and Schmitz G (2004) Loss of detoxification in inflammatory bowel disease: dysregulation of pregnane X receptor target genes. Gastroenterology 127:26-40.
Latella G, Pimpo M T, Sottili S, Zippi M, Viscido A, Chiaramonte M and Frieri G (2003) Rifaximin improves symptoms of acquired uncomplicated diverticular disease of the colon. Int J Colorectal Dis 18:55-62.
Laustsen G and Wimmett L (2005) 2004 drug approval highlights: FDA update. Nurse Pract 30:14-29; quiz 29-31.
Loguercio C, Federico A, De Girolamo V, Ferrieri A and Del Vecchio Blanco C (2003) Cyclic treatment of chronic hepatic encephalopathy with rifaximin Results of a double-blind clinical study. Minerva Gastroenterol Dietol 49:53-62.
Ma X, Shah Y, Cheung C, Guo G L, Feigenbaum L, Krausz K W, Idle J R and Gonzalez F J (2007) The PREgnane X receptor gene-humanized mouse: a model for investigating drug-drug interactions mediated by cytochromes P450 3A. Drug Metab Dispos 35:194-200.
Maher J M, Cheng X, Slitt A L, Dieter M Z and Klaassen C D (2005) Induction of the multidrug resistance-associated protein family of transporters by chemical activators of receptor-mediated pathways in mouse liver. Drug Metab Dispos 33:956-962.
Mas A, Rodes J, Sunyer L, Rodrigo L, Planas R, Vargas V, Castells L, Rodriguez-Martinez D, Fernandez-Rodriguez C, Coll I and Pardo A (2003) Comparison of rifaximin and lactitol in the treatment of acute hepatic encephalopathy: results of a randomized, double-blind, double-dummy, controlled clinical trial. J Hepatol 38:51-58.
Mild Y, Suzuki T, Tazawa C, Blumberg B and Sasano H (2005) Steroid and xenobiotic receptor (SXR), cytochrome P450 3A4 and multidrug resistance gene 1 in human adult and fetal tissues. Mol Cell Endocrinol 231:75-85.
Papi C, Koch M and Capurso L (2005) Management of diverticular disease: is there room for rifaximin? Chemotherapy 51 Suppl 1:110-114.
Pistoia M A, Lombardi L, Rossi M, Vittorini C, Cavaliere G F and Pistoia F (2004) Does rifaximin prevent complications of diverticular disease? A retrospective study. Eur Rev Med Pharmacol Sci 8:283-287.
Rosenfeld J M, Vargas R, Jr., Xie W and Evans R M (2003) Genetic profiling defines the xenobiotic gene network controlled by the nuclear receptor pregnane X receptor. Mol Endocrinol 17:1268-1282.
Saini S P, Mu Y, Gong H, Toma D, Uppal H, Ren S, Li S, Poloyac S M and Xie W (2005) Dual role of orphan nuclear receptor pregnane X receptor in bilirubin detoxification in mice. Hepatology 41:497-505.
Scarpignato C and Pelosini I (2005) Rifaximin, a poorly absorbed antibiotic: pharmacology and clinical potential. Chemotherapy 51 Suppl 1:36-66.
Shafran I and Johnson L K (2005) An open-label evaluation of rifaximin in the treatment of active Crohn's disease. Curr Med Res Opin 21:1165-1169.
Shah Y M, Ma X, Morimura K, Kim I and Gonzalez F J (2007) Pregnane X receptor activation ameliorates DSS-induced inflammatory bowel disease via inhibition of NF-{kappa}B target gene expression. Am J Physiol Gastrointest Liver Physiol 292:G1114-1122.
Sonoda J, Chong L W, Downes M, Barish G D, Coulter S, Liddle C, Lee C H and Evans R M (2005) Pregnane X receptor prevents hepatorenal toxicity from cholesterol metabolites. Proc Natl Acad Sci USA 102:2198-2203.
Staudinger J L, Goodwin B, Jones S A, Hawkins-Brown D, MacKenzie K I, LaTour A, Liu Y, Klaassen C D, Brown K K, Reinhard J, Willson T M, Koller B H and Kliewer S A (2001) The nuclear receptor PXR is a lithocholic acid sensor that protects against liver toxicity. Proc Natl Acad Sci USA 98:3369-3374.
Swales K, Kakizaki S, Yamamoto Y, Inoue K, Kobayashi K and Negishi M (2005) Novel CAR-mediated mechanism for synergistic activation of two distinct elements within the human cytochrome P450 2B6 gene in HepG2 cells. J Biol Chem 280:3458-3466.
Trapnell C B, Connolly M, Pentikis H, Forbes W P and Bettenhausen D K (2007) Absence of effect of oral rifaximin on the pharmacokinetics of ethinyl estradiol/norgestimate in healthy females. Ann Pharmacother 41:222-228.
Venturini A P (1983) Pharmacokinetics of L/105, a new rifamycin, in rats and dogs, after oral administration. Chemotherapy 29:1-3.
Wagner M, Halilbasic E, Marschall H U, Zollner G, Fickert P, Langner C, Zatloukal K, Denk H and Trauner M (2005) CAR and PXR agonists stimulate hepatic bile acid and bilirubin detoxification and elimination pathways in mice. Hepatology 42:420-430.
Yueh M F, Kawahara M and Raucy J (2005) High volume bioassays to assess CYP3A4-mediated drug interactions: induction and inhibition in a single cell line. Drug Metab Dispos 33:38-48.

What is claimed is:

1. A method of screening a compound or a salt thereof that modulates a pregnane X receptor (PXR) protein or fragment thereof, comprising contacting the PXR receptor with one or more candidate compounds, and selecting compounds or salts thereof that modulate the PXR receptor.

2. The method of claim 1, wherein the candidate compound comprises a rifamycin analog.

3. The method of claim 1, wherein modulates comprises modulating the signal transduction induced by binding of PXR protein or fragment thereof to a rifamycin analog.

4. The method of claim 1, wherein the PXR receptor is associated with a membrane, is in a transgenic mouse, is in an assay plate, is in a cell, and/or is in an artificial membrane.

5. The method of claim 1, wherein the PXR protein comprises the an amino acid sequence represented by sequence accession number O75469; Q8SQ01; Q9R1A7; O54915; NP_{—}148934; NP_{—}003880; or NP_{—}071285 or a fragment or variant thereof or a nucleic acid represented by sequence accession no NM_{—}033013; NM_{—}009803; NM_{—}022002; NM_{—}003889; CS618137; CS618135; CS618133 or a fragment or variant thereof.

6. The method of claim 1, wherein CYP3A11, GSTA1, MRP2 and OATP2 were all up-regulated.

7. The method of claim 1, further comprising pretreatment with rifaximin.

8. The method of claim 1, further comprising pretreatment with the PXR receptor protein with a candidate compound.

9. The method of claim 8, wherein pretreatment with the candidate compound does not affect the pharmacokinetics of the CYP3A substrate midazolam.

10. The method of claim 8, wherein pretreatment with the candidate compound increases a Cₘₐₓ and decreases a Tₘₐₓ of 1′-hydroxymidazolam.

11. The method of claim 1, wherein CYP3A11 increases from about 1 to about 4-fold compared to a control after treatment with the candidate compound.

12. The method of claim 1, GSTA1 mRNA is up-regulated after candidate compound treatment.

13. The method of claim 12, wherein the up-regulation ranges from between about 65% to about 200%.

14. The method of claim 1, wherein there is an up-regulation of intestinal MRP2 mRNA following candidate compound treatment.

15. A kit for screening a compound or a salt thereof that modulates a PXR receptor or fragment thereof, comprising a PXR receptor protein or an active fragment thereof and a rifamycin analog.

16. A medicament for treatment of a PXR related disorder comprising a compound or a salt thereof that modulates a pregnane X receptor (PXR) protein or fragment thereof, or a compound or its salt that modulates signal transduction induced by binding of PXR protein or fragment thereof to a rifamycin analog.

17. A method of treating, preventing, or alleviating a PXR related disorder in a subject comprising administering a compound or a salt thereof that modulates a pregnane X receptor (PXR) protein or fragment thereof.

18. The method of claim 17, wherein modulates includes modulating the signal transduction induced by binding of PXR protein or fragment thereof to a rifamycin analog.

19. The method of claim 17, wherein the PXR receptor is associated with a membrane, is in a transgenic mouse, is in an assay plate, is in a cell, and/or is in an artificial membrane.

20. The method of claim 17, wherein the PXR protein comprises the an amino acid sequence represented by sequence accession number O75469; Q8SQ01; Q9R1A7; O54915; NP_{—}148934; NP_{—}003880; or NP_{—}071285 or a fragment or variant thereof or a nucleic acid represented by sequence accession no NM_{—}033013; NM_{—}009803; NM_{—}022002; NM_{—}003889; CS618137; CS618135; CS618133 or a fragment or variant thereof.

21. A composition comprising a PXR protein agonist in an amount effective to produce a therapeutic effect.

22. A transgenic mouse comprising a homozygous disruption of the endogenous pregnane X receptor (PXR) gene.

23. The transgenic mouse of claim 22, wherein the mouse comprises a human PXR gene.

24. A cell or tissue isolated from the transgenic mouse of claim 22.

25. A method of identifying an agent capable of modulating activity of a PXR gene or of a PXR gene expression product, comprising:
administering a putative agent to the transgenic mouse of claim 23;
administering the agent to a wild-type control mouse; and
comparing a physiological response of the transgenic mouse with that of the control mouse; wherein a difference in the physiological response between the transgenic mouse and the control mouse is an indication that the agent is capable of modulating activity of the gene or gene expression product.

26. A method of screening for a drug candidate having bowel disease activity comprising:
providing a cell that expresses a PXR gene encoded by a nucleic acid sequence selected from the group consisting of the sequences NM_{—}033013; NM_{—}009803; NM_{—}022002; NM_{—}003889; CS618137; CS618135; CS618133 or. O75469; Q8SQ01; Q9R1A7; O54915; NP_{—}148934; NP_{—}003880; NP_{—}071285, or a fragments or variants thereof;
contacting the cell with a drug candidate; and
monitoring an effect of the drug candidate on an expression of the BD polynucleotide in the tissue sample.

27. An isolated antibody or antigen binding fragment thereof, that binds to a PXR polypeptide.

28. The isolated antibody of claim 27, wherein the antibody or fragment thereof is attached to a solid support; wherein the antibody is a monoclonal antibody; wherein the antibody is a polyclonal antibody; and/or wherein the antibody or fragment thereof further comprises a detectable label.
